(19) United States
(12) Patent Application Publication
(10) Pub. No.: US 2020/0024570 A1
(43) Pub. Date: Jan. 23, 2020

(54) EFFICIENT METHOD FOR SELECTION OF HIGH-PERFORMING ALGAE ISOLATES AND IDENTIFICATION OF TRAIT GENES

(71) Applicant: Board of Trustees of Michigan State University, East Lansing, MI (US)

(72) Inventors: David Kramer, Okemos, MI (US); Ben F. Lucker, Okemos, MI (US)

(21) Appl. No.: 16/445,781

(22) Filed: Jun. 19, 2019

Related U.S. Application Data
(60) Provisional application No. 62/686,939, filed on Jun. 19, 2018.

(51) Int. Cl. C12N 1/12; C12Q 1/6895

(52) U.S. Cl. CPC C12N 1/12; C12Q 2600/156; C12Q 1/6895

(57) ABSTRACT

Described herein are methods for generating robust algae strains that can grow under stressful environmental conditions.

This application claims benefit of priority to the filing date of U.S. Provisional Application Ser. No. 62/686,939, filed Jun. 19, 2018, the contents of which are specifically incorporated herein by reference in their entirety.

### GOVERNMENT FUNDING

This invention was made with government support under DE-FG02-91ER20021 awarded by the Department of Energy. The government has certain rights in the invention.

### BACKGROUND OF THE INVENTION

Despite many years of research efforts progress towards improving algal biomass productivity has been slow, particularly for complex, composite traits such as increased photosynthetic productivity, which is influenced by multiple and diverse factors that can change under different environmental conditions. The domestications of plants and animals has taken advantage of natural variations that emerged from selection for survival in diverse environmental niches. Breeding can generate novel combinations of genetic loci that not only combine multiple desirable traits, but can also result in heterosis or hybrid vigor, i.e. performance phenotypes in progeny that exceed that of their parents.

### SUMMARY

Described herein are methods for making highly productive and vigorous algae populations with rapid selection of robust individual lines. A major impediment to improving algal energy bioproduction is in delineating the complex, interacting genetic and physiological factors that contribute to productivity and resilience under diverse and often extreme environmental conditions. The methods described herein provide a solution to this problem and produce algae that exhibit strong hybrid vigor for photosynthetic productivity. The methods can include identification of genetic loci that confer favorable traits. The methods involve generating genetic diversity in an algae populations panels by crossing (mating) phenotypically-diverse algae, to thereby generate a population of one or more genetically diverse algae strains. The genetically diverse algae strains (or a population thereof) are grown under selection conditions that are environmentally-controlled and can be sufficiently stressful to generate an environmentally competitive algae population. One or more strains from an environmentally competitive algae population are quantitatively sequenced. In some cases, the entire population or pooled samples from the environmentally competitive algae population are quantitatively sequenced. Such methods can generate multiple algae strains such that a large percentage of the environmentally competitive algae population exhibits hybrid vigor under the selection conditions.

For example, a method for producing algae with strong hybrid vigor for photosynthetic productivity can involve: (a) crossing (mating) phenotypically-diverse algae strains to generate two or more genetically diverse algae strains; (b) growing one or more genetically diverse algae strain under one or more selection conditions to generate an environmentally competitive algae population; (c) measuring the photosynthetic efficiency and/or productivity of one or more algae strain of the an environmentally competitive algae population; and (d) isolating an environmentally competitive algae strain or a mixture of an environmentally competitive algae strains that exhibit hybrid vigor under the selection conditions compared to the phenotypically-diverse algae strain grown under baseline conditions. The environmentally competitive algae strains can have one genomic locus, or at least two genomic loci that provide environmental competitiveness.

Hence, also described herein are environmentally competitive algae strains having genomic loci that provide environmental competitiveness. Also described herein are mixtures of algae with at least one environmentally competitive algae strain therein, where at least one environmentally competitive algae strain has one or more that genomic locus conferring environmental competitiveness upon the algae strain(s). Algae populations that have enriched genomic loci that confer environmental competitiveness upon the population are also provided herein.

The genomic loci that confer environmental competitiveness can be isolated and incorporated into new strains of algae or into other host cells (e.g., into bacteria, yeast, fungi, insect, or algae cells for maintenance, expansion, analysis, or a combination thereof). Nucleic acids (e.g., DNA, RNA or cDNA) incorporating or encoding the environmental competitiveness genetic material can be isolated and transferred to such other host cells.

### DESCRIPTION OF THE FIGURES

FIGS. 1A-1D illustrate methods for generating and mapping algal populations that exhibit increased photosynthetic productivity and/or hybrid vigor. FIG. 1A illustrates the culture light intensity in micromole (μmol) of photosynthetically active radiation (PAR) photons per square meter per second during baseline conditions (BC) and during the light stress regime (LS). FIG. 1A also defines “gain days” and “pain days,” where the light intensity during the pain days is much lower than during the gain days. FIG. 1B illustrates F1 cross and the competition/selection methods. Parental Chlamydomonas lines CC1009 and CC2343 were crossed, and 203 F1 ml+ progeny were pooled in equal numbers and used as inoculum for cultures placed under baseline conditions that mimic a natural solar day (baseline conditions (BC), 5% CO₂ in air, 14:10 light dark cycle with zenith at noontime), hyperoxic conditions (HO, 5% CO₂ in O₂), or light stress (LS, long periods of very low light) conditions. FIG. 1C graphically illustrates the allele frequency relative to parent CC2343 (upper dashed line) of each filtered single nucleotide polymorphism (SNP) site across the genome for two independently generated inoculums (lower, darker solid line). FIG. 1D graphically illustrates the allele frequency relative to parent CC2343 (upper dashed line) across chromosome 6 for the inoculums (lower solid line) shown in FIG. 1C. The MTL region is the mating type locus (MTL) while regions 1 and 2 in FIG. 1D are potential regions for increased recombination rates.

FIGS. 2A-2C illustrate results of a fitness screen under different environmental conditions. FIG. 2A shows the daily productivity ratio of isolates under “gain days” of the light stress (LS) conditions versus baseline conditions (BC). The parental strains are identified by arrows, illustrating the difference in their productivities under light stress conditions. FIG. 2B shows the ratio of daily productivity of isolates when cultured under hyperoxic conditions (HO) vs baseline conditions (BC). The parental strains are identified by arrows, again illustrating the difference in their productivities under hyperoxic conditions. FIG. 2C shows the daily productivity in grams of ash free dry weight per square meter per day of isolates of Chlamydomonas lines CC1009 and CC2343 when cultured under baseline conditions (BC), hyperoxic conditions (HO) and light stress (LS) conditions.

FIGS. 3A-3F illustrate that environmental conditions drive population genome structure. The chromosomal numbers are shown along the x-axes. FIG. 3A shows the change in the allele frequency of the F1 baseline condition population from an inoculum relative to CC2343 (positive numbers) or CC1009 (negative numbers) after 9 (yellow in original), 21 (cream in original), 25 (violet in original), and 32 (dark blue in original) days in polyculture. As time progresses the variation in allele frequency generally increases. FIG. 3B shows the −log (p value) of the significance of the enrichment values averaged at 40 Kb windows iterated every 8 Kb across the genome for the baseline condition population after 19 days of polyculture (dataset from the F1_light stress experiment). Enriched genomic loci (EGL) results were obtained using statistical methods described in Example 1 and are presented as log-of-odds (LOD) scores, calculated as LOD=−log 10(p), where p is the probability of achieving the observed allele frequency (AF) change of a locus randomly. Regions with LOD greater than 14 (a highly restrictive cutoff) over a 60-kb window were considered to be highly statistically significant enriched genomic loci (EGLs). To illustrate the relative preference for loci from the two parents enriched genomic loci regions that represent enrichment of CC1009 were multiplied by −1. FIG. 3C shows the change in allele frequency of the F1 hyperoxic condition population for the same timepoints as FIG. 3A. FIG. 3D shows the enrichment values from the F1 hyperoxic condition population after 21 days of polyculture. FIG. 3E shows the change in allele frequency of the F1 light stress population after 6 (cream in original), 12 (violet in original) and 19 (dark blue in original) days of polyculture. FIG. 3F shows the enrichment values from the F1 hyperoxic condition population after 21 days of polyculture.

FIG. 4A-4G illustrate that F1 recombination events shape F2 population genome structures and productivity. FIG. 4A illustrates the breeding paradigm to generate the F2 progeny library. Two F1 tetrads were dissected and crossed with the two opposite mating types from the same tetrad and 30 F2 progeny from each cross were pooled to generate the F2 progeny library of about 240 lines. FIG. 4B illustrates the offset allele frequencies of chromosome 2 relative to CC2343 of chromosome 2 for the dissected tretrad progeny, which are the F1 progeny were used to generate the F2 population. The allele frequencies (AF) range from 0 to 1 and are centered on the straight horizontal dotted lines projecting from the Y-axis at a relative allele frequency of 0.5. FIG. 4C illustrates the chromosome 2 allele frequency (AF) of 240 pooled F2 lines used as the F2 inoculum. FIG. 4D illustrates the allele frequency across the genome of the F2 inoculum. FIG. 4E shows the daily productivity of the F1 generation of the co-cultured library (triangles) and the F2 generation (circles) under steady state conditions. The solid and dashed horizontal lines represent the average productivity of the parental lines CC1009 and CC2343, respectively. FIG. 4F shows the daily productivity of the parental lines CC-1009 (squares) and CC2343 (upside down triangles), F1 progeny library (right-side up triangles), and F2 progeny library (circles). FIG. 4G shows the daily productivities of parental line CC-1009 (cross-hatched bars) and parental line CC-2343 (\\\-hatched bars), compared to the F1 generation (widely ///-hatched bars) and the F2 generation (dashed hatched bars) during the high light days (gain days) of the light stress regime.

FIGS. 5A-5F illustrate histograms of the allele frequency distribution of 40 KB windows across the genome for the polyculture populations. FIG. 5A graphically illustrates the allele frequency distribution of the inoculum (dashed //-hatched bars) compared with the F1 population (solid \\-hatched bars) baseline conditions after 19 days of culture. FIG. 5B graphically illustrates the allele frequency distribution of the inoculum (dashed //-hatched bars) and the F2 population baseline conditions (solid \\-hatched bars) after 21 days of culture. FIG. 5C graphically illustrates the allele frequency distribution of the inoculum (dashed //-hatched bars) and the F1 population under hyperoxic conditions (\\-hatched bars) after 21 days of culture. FIG. 5D graphically illustrates the allele frequency distribution of the inoculum (dashed //-hatched bars) and the F2 population under hyperoxic conditions (solid \\-hatched bars) after 21 days of culture. FIG. 5E graphically illustrates the allele frequency distribution of the inoculum (dashed //-hatched bars) and the F1 light stress population (\\-hatched bars) after 19 days of culture. FIG. 5F graphically illustrates the allele frequency distribution of the inoculum (dashed //-hatched bars) and the F2 light stress population (solid \\-hatched bars) after 16 days of culture.

FIG. 6A-6C show genomic maps of daughter cells resulting from two independent meiotic events, daughters 1_1 through 1_4 are from one meiotic event and daughters 5_1 through 5_4 are from the second meiotic event. The allele frequency is relative to CC2343 and the range of each vertically varying bar in is from 0 to 1, while the straight dashed horizontal lines represent an allele frequency of 0.5.

FIG. 6A shows genomic maps of chromosomes 1-6. FIG. 6B shows genomic maps of chromosomes 7-12. FIG. 6C shows genomic maps of chromosomes 13-17.

FIG. 7A-7C illustrate that the survival of F2 progeny is heavily influenced by the F1 parental genotype. FIG. 7A shows the allele frequency distribution of the F2 baseline condition population (solid line) after 21 days of culture compared to the F1_5_4 meiotic progeny (dashed line). FIG. 7B shows the allele frequency distribution of the F2 hyperoxic condition population (solid line) after 21 days of culture, compared to the F1_1_2 meiotic progeny (dashed line). FIG. 7C shows the allele frequency distribution of the F2 light stress condition population (solid line) after 16 days of culture and the F1_5_4 (dashed --- line) and F1_5_3 (dashed and dotted line).

FIG. 8A-8C illustrates that F2 populations show a bimodal distribution of progenitor loci. The F2 library was used to inoculate triplicate ePBRs and the cultures were placed under baseline, hyperoxic, and light stress conditions. FIG. 8A shows the change of the allele frequency of the F2 baseline condition (BC) population after culture for 8 days (cream in original), 16 days (violet in original), and 21 days (dark blue in original). FIG. 8B shows the allele frequency change of the F2 hyperoxic (HO) condition population for the same timepoints as FIG. 8A. FIG. 8C shows the change in allele frequency for the F2 light stress (LS) condition population after culture for 8 days (violet in original) and 16 days (dark blue in original).

FIG. 9 illustrates that methods including breeding and selection Chlamydomonas provide high degrees of phenotypic plasticity. Step 1 involves generating genetic diversity through breeding divergent lines (e.g., in mixed cultures). Step 2 involves competition of the lines under polyculture conditions. Step 3 involves isolation and screening of the surviving progeny for increased productivity. Panel A shows isolates from the F1 baseline condition population (///-hatched bars) compared with the parental strains CC1009 and CC2343 (\\\-hatched bars for all panels). Panel B illustrates the light stress tolerance of surviving isolates of the F2 light stress population (///-hatched bars). Panel C shows the hyperoxic tolerance of F1 hyperoxic survivors (horizontally-hatched bars) compared to the parental strains (F₀; vertically-hatched bars). Panel D shows the productivity of selected progeny (///-hatched bars) compared to the parental strains (\\\-hatched bars) after an environmental simulation. Panel E shows the productivity in halotolerance media containing 20 g/L of Instant Ocean. The \\\-hatched bars show the productivity of strains isolated after hatching and selection under 20 g/L of Instant Ocean salts, the ///-hatched bars show the productivity of random F2 progeny, and the horizontally hatched bars show the productivity of the CC2343 and CC1009 strains.

FIGS. 10A-10H illustrate that populations of meiotic progeny under polyculture conditions are enriched with strains having increased fitness. FIG. 10A shows the daily productivity (in grams of ash free dry weight produced per square meter of incident light) of the progenitor lines (\\\-hatched bars) and F1 meiotic progeny (///-hatched bars) isolated after 30 days of polyculture under baseline conditions. FIG. 10B shows the average daily productivity under baseline conditions of progenitor lines (vertical hatching) and of F1 progeny (tight ///-hatching), and the productivity under hyperoxic conditions of the parental lines (wide ///-hatched bars) and F1 progeny (wide \\\-hatched bars) isolated after 30 days of polyculture under hyperoxic conditions. FIG. 10C illustrates the oxygen tolerance of the parental lines (wide ///-hatched bars) and the F1 hyperoxic condition survivors (\\\-hatched bars) in FIG. 10B. FIG. 10D shows the average daily productivity of the progenitor lines (\\\-hatched bars) and the F2 progeny (///-hatched bars) isolated after 21 days of polyculture under baseline conditions. FIG. 10E shows the average daily productivity of the progenitor lines (vertically-hatched bars) and F2 hyperoxic progeny (narrow ///-hatched bars) under baseline conditions, and of the progenitor lines (wide ///-hatched bars) and F2 hyperoxic progeny (wide \\\-hatched bars) under hyperoxic conditions isolated after 21 days of polyculture. FIG. 10F shows the oxygen tolerance of the parental lines (///-hatched bars) and the F2 hyperoxic survivors (\\\-hatched bars) in FIG. 10B. FIG. 10G shows the average daily productivity of the parental lines (narrow \\\-hatched bars) and F2 light stress progeny (narrow ///-hatched bars) under baseline conditions, and the productivity of parental lines (wide \\\-hatched bars) and F2 light stress progeny (wide ///-hatched bars) under light stress conditions when the strains were isolated after 16 days of polyculture. FIG. 10H summarizes the light stress tolerance of the lines shown in FIG. 10G, where the widely spaced ///-hatched bars represent light stress tolerance of the progenitor lines and the \\\-hatched bars represent the light stress tolerance of F2 light stress survivors. For the progeny, error bars represent the standard deviation between at least three daily growth values for selected progeny. For the parental lines, the error bars represent the standard deviation of the daily productivity values between at least three biological replicates.

FIG. 11A-11H illustrate strong heterosis persists in lines through multiple biological replicates. FIG. 11A shows the daily productivity (in grams of ash free dry weight produced per square meter of incident light) of the progenitor lines (///-hatched bars) and choice F1 meiotic progeny (\\\-hatched bars) isolated after 30 days of polyculture under baseline conditions. FIG. 11B shows the average daily productivity under baseline conditions of the parental lines (narrow ///-hatched bars) and choice F1 progeny (narrow \\\-hatched bars) as well as under hyperoxic conditions of the parental lines (widely \\\-hatched bars) and choice F1 progeny (widely ///-hatched bars) isolated after 30 days of polyculture under hyperoxic conditions. FIG. 11C shows the oxygen tolerance of the parental lines ((///-hatched bars) and the selected F1 hyperoxic survivors (\\\-hatched bars) from the results shown in FIG. 11B. FIG. 11D shows the productivity of the progenitor lines (///-hatched bars) and selected F2 baseline survivors (\\\-hatched bars) isolated after 21 days of polyculture under baseline conditions. FIG. 11E shows the average daily productivity of the parental lines (narrowly ///-hatched bars) selected F2 hyperoxic progeny (narrowly \\\-hatched bars) after 21 days of polyculture under baseline conditions, as well as the productivity of the progenitor lines (widely \\\-hatched bars) and selected F2 hyperoxic progeny (wide ///-hatched bars) isolated after 21 days of polyculture under hyperoxic conditions. FIG. 11F shows the oxygen tolerance of the parental lines (///-hatched bars) and the selected F2 hyperoxic survivors shown (\\\-hatched bars) in FIG. 11E. FIG. 11G shows the average daily productivity of the parental lines (narrow ///-hatched bars) and chosen F2 light stress progeny (narrow \\\-hatched bars) isolated after 16 days of polyculture under baseline conditions as well as the average daily productivity of the parental lines (wide \\\-hatched bars) and chosen F2 light stress progeny (wide ///-hatched bars) isolated after 16 days of polyculture under light stress conditions. FIG. 11H summarizes the light stress tolerance of the lines shown in FIG. 11G, ///-hatched bars represent the progenitor lines and \\\-hatched bars represent F2 light stress survivors. Error bars represent standard deviation of the average daily growth from a minimum of three biological replicates. Asterisks denotes a maximum p-value of 0.05 from a two-tailed t-Test while double crosses represent a maximum p-value of 2^{e-5}.

FIG. 12 illustrates the light intensity (solid line) and temperature (dashed line) during an environmental simulation selection.

### DETAILED DESCRIPTION

Methods are described herein for generating algal strains that exhibit increased fitness or productivity over the progenitor strains. The methods can include mapping of the genetic loci that provide the increased productivity. These methods can generate large populations of genetically diverse algae and can rapidly reduce the population diversity by selecting for strains with increased fitness.

For example, one method for producing algae with strong hybrid vigor for photosynthetic productivity can involve: (a) crossing (mating) phenotypically-diverse algae strains to generate two or more genetically diverse algae strains; (b) growing one or more genetically diverse algae strain under one or more selection conditions to generate an environmentally competitive algae population; (c) measuring the photosynthetic efficiency and/or productivity of one or more algae strain of the an environmentally competitive algae population; and (d) isolating an environmentally competitive algae strain or a mixture of an environmentally competitive algae strains that exhibit hybrid vigor under the selection conditions compared to the phenotypically-diverse algae strain grown under baseline conditions.

### Algae

As used herein, the term “algae” may mean any type of microalgae or macroalgae. For example, an algae strain can be any sexually reproductive type of algae. In some cases, the term means algae species of the genus of Protococcus, Ulva, Codium, Pheodactylum, Enteromorpha, Neochloris and/or Chlamydomonas. In some cases, the algae species is a species of algae. The algal species can also be able to mate. For example, algae species can form gametes that then fuse to form a zygote. In some cases, the algae species can be a Chlamydomonas species. Chlamydomonas is a genus of green algae consisting of about 325 species of unicellular flagellates, found in stagnant water and on damp soil, in freshwater, seawater, and even in snow. In some cases, the algae species can be Chlamydomonas reinhardtii.

Algae may be collected in fresh water or salt water shores, or soils. For example, various species of the genii Protococcus, Ulva, Codium and Entemmorpha can be collected from fresh water and salt water sources in Salisbury, Md., Assateague National Seashore and at Ocean City, Md. In some cases, the algae species Algae species can also be obtained from the Chlamydomonas Resource Center (see, website at www.chlamycollection.org).

The most widely used laboratory species is Chlamydomonas reinhardtii (Dang). The wild-type of this species (strain 137C) was isolated from soil by Dr. Smith in 1948 in USA (see in rf. Levine 1960). Cells of this wild-type strain are haploid and can grow on a simple medium of inorganic salts, using photosynthesis to provide energy. Cells can also grow in total darkness when acetate is provided as an alternative carbon source. When deprived of nitrogen, haploid cells of opposite mating types can fuse to form a diploid zygospore which forms a hard, outer-wall that protects it from adverse environmental conditions. When conditions improve (e.g. when nitrogen is restored to the culture medium), the diploid zygote undergoes meiosis and releases four haploid cells that resume the vegetative life cycle.

In some cases, Chlamydomonas strains CC1009 (mt−) and CC2343 (mt+) can be used. These strains can be obtained from the Chlamydomonas Resource Center (see, website at www.chlamycollection.org/product/cc-1009-wild-type-mt-utex-89/ and www.chlamycollection.org/product/cc-2343-wild-type-mt-jarvik-224-melbourne-fl/).

As used herein “phenotypically-diverse” means that two or more algal strains exhibits different responses to environmental conditions. In some cases, phenotypically-diverse algal strains exhibit different productivities under the same environmental conditions, where for example the productivities are daily productivities. The productivities of algal strains can be measured as grams of ash free dry weight of each algae strain per square meter per day. In some cases, the productivities can be measured as chlorophyll concentration of each algae strain per square meter per day.

Parental strains for mating can in some cases be selected that exhibit differences in their productivities under different environmental conditions. For example, a first algae strain may exhibit 50% (or 20%, or 30%, or 40%, or 60%, or 70%, or 80%) higher productivity under a first environmental condition than a second algae strain. However, the second algae strain may exhibit 50% (or 20%, or 30%, or 40%, or 60%, or 70%, or 80%) higher productivity under a second environmental condition than the first algae strain. The first and second strains may, for example, be selected as parental strains for crossing because they exhibit useful phenotypically-diverse characteristics that could be genetically transmitted to their progeny.

Hence, two algae strains that exhibit at least one phenotypically-diverse trait can be selected as parent strains. In some cases, the selected parental strains exhibit at least two, or at least three, or at least four, or at least five phenotypically-diverse traits. Parental strains can be selected that exhibit a propensity to survive (e.g., are productive) under selection environmental conditions such as increased oxygen atmosphere, a reduced carbon dioxide atmosphere, reduced light conditions, increased light conditions, increased salt conditions, increased temperatures, decreased temperatures, fluctuating temperatures, reduced nitrogen conditions, reduced pH conditions, increased pH conditions, conditions comprising macronutrients, conditions comprising micronutrients, conditions comprising pollutants, reduced phosphate conditions, or increased phosphate conditions.

Progeny of such parents are selected that exhibit at least equivalent productivities, or more preferably, even higher productivities under any of the selection environmental conditions than either of their parental strains. Such progeny are thus environmentally competitive. For example, the progeny can exhibit at least 5%, or at least 10%, or at least 20%, or at least 30%, or at least 40%, or at least 50%, or at least 60%, or at least 70%, or at least 80%, or at least 90%, or at least 100%, or at least 150% higher productivity than either of the parental strains. The productivities of progeny can be increased from one generation to another generation, and over multiple generations, to yield progeny strains with desired high levels of productivities and environmental competitiveness.

### Algae Maintenance Culture

Algae can be maintained under a variety of conditions. For example, algae cultures can be maintained on Sueoka's high salt media (Sueoka, Proc. Natl. Acad. Sci. USA 46, 83-91 (1960) or 2NBH media, which is a Bristol media (available at the website utex.org/products/bristol-medium) with twice the amount of sodium nitrate. The media can also contain Hutner's trace elements (Hutner et al., Proc. Am. Philos. Soc. 94: 152-170 (1950), see website at chlamycollection.org/methods/media-recipes/hutners-trace-elements/).

A stationary culture method can be used as for culture of algae, but a shaking culture method or a deep aeration stirring culture method can also be used for culturing algae. The shaking culture may be reciprocal shaking or rotary shaking. The algae can be cultured at a temperature of 15° C. to 40° C. In some cases, the cultures can be maintained at room temperature.

In some cases, the algae can be grown or maintained in environmental photobioreactors (ePBRs), for example, as described in Lucker et al. Algal Research, 6, Part B, 242-249 (2014).

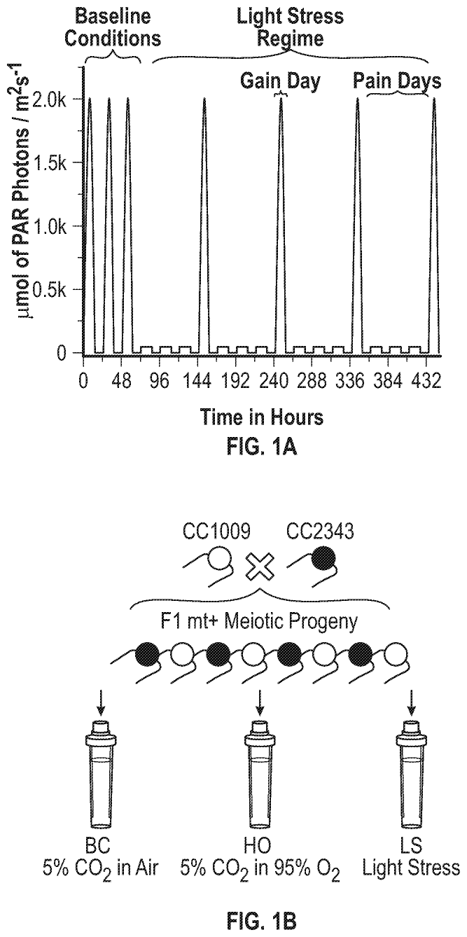

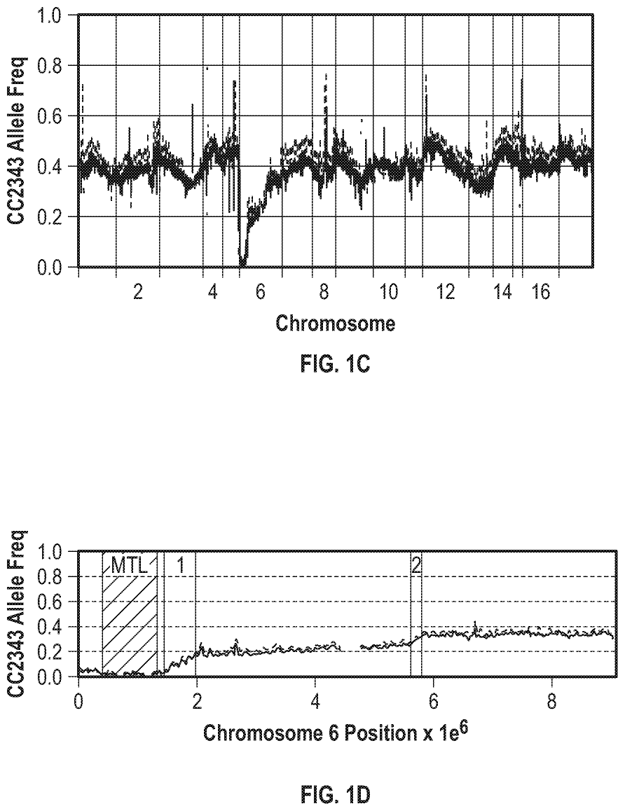

Baseline conditions can be used as control conditions that mimic a natural solar day. These conditions can include culturing in 5% carbon dioxide in air, using a 14 hour:10-hour light:dark cycle. The 14:10 hour (light:dark) diurnal cycle can simulate a cloudless day, with light intensity ascending to a zenith with maximum photosynthetically active radiation (PAR) of about 2000 μmol photons per square meter per second, and descending until dark, delivered in a sinusoidal form, as illustrated in the inset to FIG. 1C.

### Selective Culture Conditions

Algae can be subjected to culture conditions to select for increased productivity (or competitive fitness). For example, algae can be cultured under selective conditions that include increased oxygen (e.g., an atmosphere that contains more than 21% oxygen), reduced or increased carbon dioxide (e.g., an atmosphere with less or more than 0.04%), reduced light conditions (e.g., less than 2000 μmol photons per square meter per second), increased light conditions (e.g., more than 2000 μmol photons per square meter per second), increased salt conditions (e.g., more than 0.4 mM sodium chloride), increased temperatures (e.g., more than 40° C.), decreased temperatures (e.g., less than 15° C.), fluctuating temperatures (e.g., fluctuating between 12 and 44° C.), reduced nitrogen conditions (e.g., less than 0.002 mM nitrate, urea, or ammonia), increased nitrogen conditions (e.g., more than 0.002 mM nitrate, urea, or ammonia), reduced pH conditions (e.g., less than pH 7.5), increased pH conditions (e.g., greater than pH 7.5), conditions comprising various macronutrients (e.g., increased or decreased concentrations of potassium, calcium, sulfur, magnesium, or combinations thereof), conditions comprising various micronutrients (e.g., increased or decreased concentrations of iron, boron, chlorine, manganese, zinc, copper, molybdenum, nickel or combinations thereof), conditions comprising pollutants (e.g., heavy metals, gold, cobalt, lead, arsenic, cadmium, chromium strontium, or mercury; detergents, insecticides, fertilizers, herbicides, hydraulic fracturing fluids, petroleum, gasoline, oil, or combinations thereof), reduced phosphate conditions (e.g., less than 1 mM), increased phosphate conditions (e.g., more than 1 mM), or combinations thereof.

For example, algae can be cultured under conditions that include increased oxygen, which can include an atmosphere that contains more than 21% oxygen, more than 30% oxygen, more than 40% oxygen, more than 50% oxygen, more than 60% oxygen, more than 70% oxygen, more than 80% oxygen, more than 90% oxygen. In some cases, algae can be cultured under conditions that include 5% carbon dioxide in an oxygen atmosphere (hyperoxic or HO conditions).

For example, algae can be cultured under conditions that include reduced carbon dioxide, which can include an atmosphere with less than 0.04%, or less than 0.5%, or less than 1%, or less than 2%, or less than 5% carbon dioxide.

For example, algae can be cultured under conditions that include reduced light conditions, which can include illumination at less than 2000 μphotons per square meter per second, less than 1000 μmol photons per square meter per second, less than 500 μmol photons per square meter per second, less than 250 μmol photons per square meter per second, less than 100 μmol photons per square meter per second, less than 75 μmol photons per square meter per second. In some cases, algae can be cultured under conditions that include alternating periods of time of normal illumination (e.g., about 2000 μmol photons per square meter per second) and reduced light conditions illumination (e.g., about 50 μmol photons per square meter per second). Each period of illumination can be about 1-3 days of a light:dark cycle, where the light cycle is about 10-14 hours of either normal illumination or reduced illumination. For example, the algae can be cultured under light stress (LS) conditions with 1-3 days of normal illumination alternated with a series of 1-3 “light starvation” days, which consisted of a 14:10 hour rectangular wave with a PAR intensity of 50 μmol photons per square meter per second.

For example, algae can be cultured under conditions that include increased light conditions, which can include illumination at more than 2000 μmol photons per square meter per second, more than 2200 μmol photons per square meter per second, more than 2500 μmol photons per square meter per second, more than 3000 μmol photons per square meter per second, more than 3500 μmol photons per square meter per second, more than 4000 μmol photons per square meter per second, or more than 5000 μmol photons per square meter per second. Such culture under conditions that include increased light conditions can be either continuous exposure to increased light conditions or use of alternating periods of time of normal illumination (e.g., about 2000 Iμmol photons per square meter per second) and increased light conditions.

For example, algae can be cultured under conditions that include increased salt conditions, which can include culturing the algae in more than 0.0004 M sodium chloride, more than 0.005 M sodium chloride, more than 0.01 M sodium chloride, more than 0.05 M sodium chloride, more than 0.1 M sodium chloride, more than 0.2 M sodium chloride, or more than 0.3M. In some cases, the algae can be cultured under conditions that include about 0.34 M (e.g., 20 g/L NaCl).

For example, algae can be cultured under conditions that include increased temperatures, which can include culturing the algae at more than 40° C., more than 41° C., more than 42° C., more than 43° C., more than 44° C., more than 45° C., more than 46° C., more than 47° C., more than 48° C., more than 49° C. or more than 50° C. In some cases, algae can be cultured under conditions that include fluctuating temperatures (e.g., fluctuating between 12 and 44° C.). Such fluctuation can include culturing a selected temperature for 1-14 hours, or for 1-3 days, or for 1-7 days.

For example, algae can be cultured under conditions that include decreased temperatures, which can include culturing the algae at less than 15° C., at less than 14° C., at less than 13° C., at less than 12° C., at less than 11° C., at less than 10° C., at less than 7° C., at less than 5° C., at less than 4° C., at less than 2° C., at less than 1° C., or at less than 0° C. Such fluctuation can include culturing a selected temperature for 1-14 hours, or for 1-3 days, or for 1-7 days.

For example, algae can be cultured under conditions that include reduced nitrogen conditions, which can include culturing the algae at less than 0.2 mM nitrate, less than 0.01 mM nitrate, less than 0.005 mM nitrate, less than 0.001 mM nitrate, less than 0.00001 mM nitrate, or at about 0 mM nitrate.

For example, algae can be cultured under conditions that include increased nitrogen conditions, which can include culturing the algae at more than 0.2 mM nitrate, more than 0.3 mM nitrate, more than 0.5 mM nitrate, more than 1 mM nitrate, more than 2 mM, more than 3.5 mM nitrate, more than 4.0 mM nitrate, more than 5.0 mM nitrate, more than 10 mM nitrate, more than 20 mM nitrate, more than 30 mM nitrate, more than 50 mM nitrate, or more than 100 mM nitrate.

For example, algae can be cultured under conditions that include reduced pH conditions, which can include culturing the algae in a medium with a pH that is less than pH 7.5, or less than pH 7.4, or less than pH 7.3, or less than pH 7.2, or less than pH 7.1, or less than pH 7.0, or less than pH 6.9, or less than pH 6.8, or less than pH 6.7, or less than pH 6.6, or less than pH 6.5, or less than pH 6.3, or less than pH 6.0, or less than pH 5.8, or less than pH 5.5.

For example, algae can be cultured under conditions that include increased pH conditions, which can include culturing the algae in a medium with a pH that is greater than pH 7.2, or greater than pH 7.3, or greater than pH 7.4, or greater than pH 7.5, or greater than pH 7.6, or greater than pH 7.7, or greater than pH 7.8, or greater than pH 7.9, or greater than pH 8.0, or greater than pH 8.2, or greater than pH 8.3, or greater than pH 8.4, or greater than pH 8.5, or greater than pH 8.7, or greater than 9.0.

For example, algae can be cultured under conditions that include pollutants such as heavy metals, detergents, insecticides, fertilizers, herbicides, hydraulic fracturing fluids, petroleum, gasoline, oil, or combinations thereof.

For example, algae can be cultured under conditions that include reduced phosphate conditions, which can include culturing the algae at less than 1 mM phosphate, or less than 0.5 mM phosphate, or less than 0.1 mM phosphate, or less than 0.05 mM phosphate, or less than 0.01 mM phosphate, or less than 0.005 mM phosphate, or less than 0.001 mM phosphate, or 0 mM phosphate.

For example, algae can be cultured under conditions that include increased phosphate conditions, which can include culturing the algae at more than 1 mM, more than 2 mM phosphate, more than 3 mM phosphate, more than 5 mM phosphate, more than 7 mM phosphate, more than 10 mM phosphate, more than 20 mM phosphate, more than 50 mM phosphate, more than 70 mM phosphate, more than 100 mM phosphate, more than 150 mM phosphate.

Controlled and reproducible conditions can be obtained by use of environmental photobioreactors (ePBRs) (Lucker et al. Algal Research, 6, Part B, 242-249 (2014)) under turbidostat control with dilution of the culture when the measured turbidity raises above a set point. The turbidity of the culture can be measured at various intervals, and the culture can be diluted with fresh medium to reduce the number of algae cells, or to maintain a constant chlorophyll concentration within the culture of between 4 and 5 μg chlorophyll per milliliter.

### Measuring Algae Productivity (Vigor)

The productivity or vigor of a mixed or pure algae culture can be measured in a variety of ways.

For example, the productivity or vigor of an algae culture can be measured by the number of daily dilutions (e.g. of 5 or 10 ml) needed to maintain the turbidity or chlorophyll content at constant level.

In another example, the ash free dry weight (AFDW) can be used to measure the productivity or vigor of a mixed or pure algae culture. For example, an aliquot of the algae can be collected and dried, then divided by the volume or the cross-section area of the culture vessel at 15 cm (0.002687 m²). For example, the ash free dry weight can be determined by passing an aliquot of the algae culture through a filter and drying the retained matter (algae) over night at 104° C. prior to weighing to obtain the dry weight. This weight can contain non-organic solids (e.g., metals and a filter if the filter is a glass filter). The weight of these non-organic solids (referred to as the ash weight) can be deducted from the dry weight to obtain the ash free dry weight (AFDW). To obtain the ash weight, the organic matter can be removed from the filter by heating the samples to 550° C. for a minimum of 30 minutes prior to weighing the sample for the “ash weight.” The AFDW is the dry weight minus the ash weight.

The populations of environmentally competitive algae, and/or isolated environmentally competitive algae strains, can exhibit at least one, or at least two, or at least three, or at least four, or at least five, or at least seven, or at least eight, or at least ten, or at least twelve, or at least fifteen, or at least seventeen, or at least twenty more daily dilutions than the phenotypically-diverse algae parental strains grown under the same conditions (e.g., under selective culture conditions).

The populations of environmentally competitive algae, and/or isolated environmentally competitive algae strains, can provide at least 2%, or at least 3%, or at least 5%, or at least 7%, or at least 8%, or at least 9%, or at least 10%, or at least 12%, or at least 13%, or at least 15%, or at least 17%, or at least 20%, or at least 25%, or at least 30%, or at least 40%, or at least 50%, or at least 75%, or at least 80%, or at least 90%, or at least 95% more ash free dry weight (AFDW) than the phenotypically-diverse algae parental strains grown under the same conditions (e.g., under selective culture conditions). In some cases, the populations of environmentally competitive algae, and/or isolated environmentally competitive algae strains, can provide at least 2-fold, or at least 3-fold, or at least 5-fold, or at least 7-fold, or at least 10-fold, or at least 15-fold, or at least 20-fold more ash free dry weight (AFDW) than the phenotypically-diverse algae parental strains grown under the same conditions (e.g., under selective culture conditions).

The populations of environmentally competitive algae, and/or isolated environmentally competitive algae strains, exhibit increased vigor as described herein compared to one or more parental strains.

### Environmentally Competitive Algae

The methods described herein can generate populations of environmentally competitive algae, and isolated environmentally competitive algae strains, that can survive and grow under conditions that include increased oxygen (e.g., an atmosphere that contains more than 21% oxygen), reduced carbon dioxide (e.g., an atmosphere with less than 0.04%), reduced light conditions (e.g., less than 2000 μmol photons per square meter per second), increased light conditions (e.g., more than 2000 μmol photons per square meter per second), increased salt conditions (e.g., more than 0.4 mM sodium chloride), increased temperatures (e.g., more than 40° C.), decreased temperatures (e.g., less than 15° C.), fluctuating temperatures (e.g., fluctuating between 12 and 44° C.), reduced nitrogen conditions (e.g., less than 0.002 mM nitrate), reduced phosphate conditions (e.g., less than 1 mM), or increased phosphate conditions (e.g., more than 1 mM).

For example, methods described herein can generate populations of environmentally competitive algae, and isolated environmentally competitive algae strains, that can survive and grow under conditions that include an atmosphere that contains more than 21% oxygen, more than 30% oxygen, more than 40% oxygen, more than 50% oxygen, more than 60% oxygen, more than 70% oxygen, more than 80% oxygen, more than 90% oxygen. In some cases, algae can be cultured under conditions that include 5% carbon dioxide in an oxygen atmosphere (hyperoxic or HO conditions).

For example, methods described herein can generate populations of environmentally competitive algae, and isolated environmentally competitive algae strains, that can survive and grow under conditions that include reduced carbon dioxide, which can include an atmosphere with less than 0.04%, or less than 0.5%, or less than 1%, or less than 2%, or less than 5% carbon dioxide.

For example, methods described herein can generate populations of environmentally competitive algae, and isolated environmentally competitive algae strains, that can survive and grow under conditions that include illumination at less than 2000 μmol photons per square meter per second, less than 1000 μmol photons per square meter per second, less than 500 μmol photons per square meter per second, less than 250 μmol photons per square meter per second, less than 100 μmol photons per square meter per second, less than 75 μmol photons per square meter per second. In some cases, the populations of environmentally competitive algae, and isolated environmentally competitive algae strains, that can survive and grow under conditions that include alternating periods of time of normal illumination (e.g., about 2000 μmol photons per square meter per second) and reduced light conditions illumination (e.g., about 50 μmol photons per square meter per second). Each period of illumination can be about 1-3 days of a light:dark cycle, where the light cycle is about 10-14 hours of either normal illumination or reduced illumination. For example, the populations of environmentally competitive algae, and isolated environmentally competitive algae strains, that can survive and grow under light stress (LS) conditions with 1-3 days of normal illumination alternated with a series of 1-3 “light starvation” days, which consisted of a 14:10 hour rectangular wave with a PAR intensity of 50 μmol photons per square meter per second.

For example, methods described herein can generate populations of environmentally competitive algae, and isolated environmentally competitive algae strains, that can survive and grow under conditions that include illumination at more than 2000 μmol photons per square meter per second, more than 2200 μmol photons per square meter per second, more than 2500 μmol photons per square meter per second, more than 3000 μmol photons per square meter per second, more than 3500 μmol photons per square meter per second, more than 4000 μmol photons per square meter per second, or more than 5000 mol photons per square meter per second. Such populations of environmentally competitive algae, and isolated environmentally competitive algae strains, that can survive and grow under either continuous exposure to increased light conditions or under alternating periods of time of normal illumination (e.g., about 2000 μmol photons per square meter per second) and increased light conditions.

For example, methods described herein can generate populations of environmentally competitive algae, and isolated environmentally competitive algae strains, that can survive and grow under conditions that include more than 0.0004 M sodium chloride, more than 0.005 M sodium chloride, more than 0.01 M sodium chloride, more than 0.05 M sodium chloride, more than 0.1 M sodium chloride, more than 0.2 M sodium chloride, or more than 0.3M. In some cases, the populations of environmentally competitive algae, and isolated environmentally competitive algae strains, can survive and grow under conditions that include about 0.34 M (e.g., 20 g/L NaCl).

For example, methods described herein can generate populations of environmentally competitive algae, and isolated environmentally competitive algae strains, that can survive and grow under conditions that include culturing the algae at more than 40° C., more than 41° C., more than 42° C., more than 43° C., more than 44° C., more than 45° C., more than 46° C., more than 47° C., more than 48° C., more than 49° C., or more than 50° C. In some cases, populations of environmentally competitive algae, and isolated environmentally competitive algae strains, can be cultured under conditions that include fluctuating temperatures (e.g., fluctuating between 12 and 44° C.). Such fluctuation can include culturing a selected temperature for 1-14 hours, or for 1-3 days, or for 1-7 days.

For example, methods described herein can generate populations of environmentally competitive algae, and isolated environmentally competitive algae strains, that can survive and grow under conditions that include culturing the algae at less than 15° C., at less than 14° C., at less than 13° C., at less than 12° C., at less than 11° C., at less than 10° C., at less than 7° C., at less than 5° C., at less than 4° C., at less than 2° C., at less than 1° C., or at less than 0° C. Such fluctuation can include culturing at a selected temperature for 1-14 hours, or for 1-3 days, or for 1-7 days.

For example, methods described herein can generate populations of environmentally competitive algae, and isolated environmentally competitive algae strains, that can survive and grow under conditions that include culturing the algae at less than 0.2 mM nitrate, less than 0.01 mM nitrate, less than 0.005 mM nitrate, less than 0.001 mM nitrate, less than 0.00001 mM nitrate, or at about 0 mM nitrate.

For example, methods described herein can generate populations of environmentally competitive algae, and isolated environmentally competitive algae strains, that can survive and grow under conditions that include culturing the algae at more than 0.2 mM nitrate, more than 0.3 mM nitrate, more than 0.5 mM nitrate, more than 1 mM nitrate, more than 2 mM, more than 3.5 mM nitrate, more than 4.0 mM nitrate, more than 5.0 mM nitrate, more than 10 mM nitrate, more than 20 mM nitrate, more than 30 mM nitrate, more than 50 mM nitrate, or more than 100 mM nitrate.

For example, methods described herein can generate populations of environmentally competitive algae, and isolated environmentally competitive algae strains, that can survive and grow under conditions that include culturing the algae at less than 1 mM phosphate, or less than 0.5 mM phosphate, or less than 0.1 mM phosphate, or less than 0.05 mM phosphate, or less than 0.01 mM phosphate, or less than 0.005 mM phosphate, or less than 0.001 mM phosphate, or 0 mM phosphate.

For example, methods described herein can generate populations of environmentally competitive algae, and isolated environmentally competitive algae strains, that can survive and grow under conditions that include culturing the algae at more than 1 mM, more than 2 mM phosphate, more than 3 mM phosphate, more than 5 mM phosphate, more than 7 mM phosphate, more than 10 mM phosphate, more than 20 mM phosphate, more than 50 mM phosphate, more than 70 mM phosphate, more than 100 mM phosphate, more than 150 mM phosphate.

The populations of environmentally competitive algae can include a variety of environmentally competitive algae strains. But the populations of environmentally competitive algae can also contain some algae strains that are not particularly environmentally competitive. For example, the populations of environmentally competitive algae can include at least 10%, or at least 20%, or at least 30%, or at least 40%, or at least 50%, or at least 60%, or at least 70%, or at least 80%, or at least 90%, or at least 95%, or at least 96%, or at least 97%, or at least 98%, or at least 99%, or at least 99.5% environmentally competitive algae under any of the environmentally stressful conditions described herein.

The populations of environmentally competitive algae, and/or isolated environmentally competitive algae strains, exhibit increased vigor as described herein compared to one or more parental strains.

Such populations of environmentally competitive algae, or isolated environmentally competitive algae strains, can have one or more genomic locus that confers resistance or the ability to compete under such environmentally stressful conditions. In some cases, the populations of environmentally competitive algae, or isolated environmentally competitive algae strains, can have two or more, three or more, four or more, five or more, six or more, seven or more, eight or more, nine or more, ten or more, twelve or more, fifteen or more, or twenty or more genomic loci that confer resistance or the ability to compete under such environmentally stressful conditions.

The environmentally competitive algae strains can have one genomic locus, or at least two genomic loci that provide environmental competitiveness. Also described herein are mixtures of algae with at least one environmentally competitive algae strain that has one or more that genomic locus conferring environmental competitiveness upon the algae strain (a). Algae populations that have enriched genomic loci that confer environmental competitiveness upon the population are also provided herein.

The genomic loci that provide environmental competitiveness can be isolated, recombinantly replicated in plasmids, and/or incorporated into expression vectors with heterologous regulatory elements such as promoters and terminators that facilitate expression. The genomic loci that provide environmental competitiveness can also be introduced into other strains of algae.

### Sequencing

In some cases, it can be useful to sequence genomic DNA, RNA or cDNA of genetically diverse algae strain(s), for example, from genetically diverse algae strain(s) that exhibit improved productivity or vigor. Such sequencing can be performed on isolated algae strains, or on mixtures of algae. The sequencing can identify the genomic loci that confer environmental competitiveness, resistance to environmentally stressful conditions, or the ability to compete under such environmentally stressful conditions. Strains with identified genomic loci that confer resistance or the ability to compete under such environmentally stressful conditions can be isolated and expanded to provide a population of isogenic environmentally competitive algae.

Sequencing analysis can involve the use of any convenient method. In some cases, the sequencing can be performed as ultra-deep sequencing, such as described in Marguiles et al., Nature 437 (7057): 376-80 (2005). Briefly, segments of the algae nucleic acids can be amplified to provide a pool of DNA amplicons. The amplicons can be diluted and mixed with beads such that each bead captures a single molecule of the amplified DNA. The DNA molecule on each bead is then amplified to generate millions of copies of the sequence which all remain bound to the bead. Such amplification can occur by PCR. Each bead can be placed in a separate well, which can be a (optionally addressable) picoliter-sized well. In some cases, each bead can be captured within a droplet of a PCR-reaction-mixture-in-oil-emulsion and PCR amplification can occur within each droplet. The amplification on the bead results in each bead carrying at least one million, at least 5 million, or at least 10 million copies of the original amplicon coupled to it. Finally, the beads are placed into a highly parallel sequencing by synthesis machine which generates over 400,000 reads (about 100 bp per read) in a single 4-hour run. Other methods for ultra-deep sequencing that can be used are described in Hong, S. et al. Nat. Biotechnol. 22(4):435-9 (2004); Bennett. B. et al. Pharmacogenomics 6(4):373-82 (2005); Shendure, P. et al. Science 309 (5741):1728-32 (2005).

The nucleic acid segments selected for sequencing can vary. In some cases, the segments can include a site that has a single nucleotide polymorphism (SNP) in the species of algae selected. For example, as described in the Examples, a list of mapped SNPs unique to Chlamydomonas strains CR1009 or CR2343 can be used to assess whether a given SNP is present in a selected genetically diverse algae strain or in a mixture of genetically diverse algae strain(s). Comparison of the incidence or frequency of SNPs in the genetically diverse algae strain(s) to their parental strain(s) provides an indication of the extent to which the genetically diverse algae strain(s) deviate genetically from the parent strains.

In some cases, allele frequencies can be determined by adjacent averaging all SNP frequencies using selected segments (windows) of genomic windows and repeating the window every 8 Kb down each chromosome. To determine regions of the genome with significant changes in SNP frequency for each selection condition and assay time-point, the frequencies of markers attributable to parent or genetically diverse algae strain(s) for each chromosome can be determined. The statistical significance of differences between any pair of samples can be calculated. Enriched genomic loci (EGLs) can be identified in genetically diverse algae strains as regions of the genome whose average p-value for difference from parent sequence is significant.

Strains with identified genomic loci that confer resistance or the ability to compete under such environmentally stressful conditions can be isolated and expanded to provide a population of isogenic environmentally competitive algae. In some cases, it can be useful to generate mixtures of algae strains, where the different strains are resistance or exhibit the ability to compete under different environmentally stressful conditions.

DNA (e.g., genomic or cDNA) that confers environmental competitiveness can be isolated and maintained in a convenient host cell. Such host cells can be bacterial, fungal, insect, plant, or algae host cells.

### Definitions

Hybrid vigor, also called heterosis or outbreeding enhancement, is the improved or increased function of any biological quality in a hybrid offspring.

The photosynthetic efficiency is the fraction of light energy converted into chemical energy during photosynthesis in plants and algae. Photosynthesis can be described by the simplified chemical reaction

6H₂O+6CO₂+energy→C₆H₁₂O₆+6O₂

where C₆H₁₂O₆ is glucose (which is subsequently transformed into other sugars, cellulose, lignin, and so forth). The value of the photosynthetic efficiency relates to how light energy is defined and depends on whether only the light that is absorbed is counted, and on what kind of light is used. In general, it takes at least eight photons, or nine photons, or ten photons, or eleven photons, or twelve photons to utilize one molecule of CO₂. The Gibbs free energy for converting a mole of CO₂ to glucose is 114 kcal, whereas eight moles of photons of wavelength 600 nm contains 381 kcal, giving a nominal efficiency of 30%. However, photosynthesis can occur with light up to wavelength 720 nm so long as there is also light at wavelengths below 680 nm to keep Photosystem II operating. Using longer wavelengths means less light energy is needed for the same number of photons and therefore for the same amount of photosynthesis. For actual sunlight, where only 45% of the light is in the photosynthetically active wavelength range, the theoretical maximum efficiency of solar energy conversion is approximately 11%. However, plants do not absorb all incoming sunlight (due to reflection, respiration requirements of photosynthesis and the need for optimal solar radiation levels) and do not convert all harvested energy into biomass, which results in an overall photosynthetic efficiency of 3 to 6% of total solar radiation. If photosynthesis is inefficient, excess light energy must be dissipated to avoid damaging the photosynthetic apparatus. Energy can be dissipated as heat (non-photochemical quenching) or emitted as chlorophyll fluorescence.

The following Examples illustrate experimental work performed in the development of the methods and strains described herein.

### Example 1: Materials and Methods

This Example describes some of the materials and methods used in the development of the inventive algae strains and methods.

### Strains, Media and Generation of Progeny

Chlamydomonas strains CC1009 (mt−) and CC2343 (mt+) were obtained through the Chlamydomonas Resource Center (see, website at www.chlamycollection.org/product/cc-1009-wild-type-mt-utex-89/ and www.chlamycollection.org/product/cc-2343-wild-type-mt-jarvik-224-melbourne-fl/).

CC1009 and CC2343 cells were crossed to generate approximately 20° F.1 mt-progeny. The 246 F2 progeny population was generated by dissecting two F1 zygotes and crossing the reciprocal mating types of each tetrad (each mt− with mt+ from each tetrad) for total of 8 F1 crosses (˜30 lines from each cross). Cultures were maintained on either Sueoka's high salt media (Sueoka, 1960) or 2NBH media, which is a Bristol media with 2× sodium nitrate and Hutner's trace elements added (Davey et al 2012).

### Growth and Competition Conditions

To achieve highly controlled and reproducible conditions environmental photobioreactors (ePBRs) were used (Lucker and Hall et al. 2014) under turbidostat control that diluted the culture when the measured turbidity rose above a set point. At ten-minute measuring intervals, cultures with turbidity above the setpoint were diluted with 5 mL of fresh medium, until the turbidity decreased below the setpoint. In this way, the relative biomass growth for the cultures over a time range could be roughly estimated by the number of dilutions, as described in the following section (see also Lucker and Hall et al. 2014). For these experiments, the set point was adjusted to maintain a constant chlorophyll concentration between 4 and 5 pg chlorophyll per milliliter. The ePBR culture height was set to 15 cm using a volume 330 ml of 2NBH media. For individual phenotyping conditions, cultures were pre-conditioned to grown in ePBRs to a chlorophyll 4 pg per ml and maintained in turbidistat mode using the standard light conditions for at least 3 days prior to measuring productivity.

Strains of Chlamydomonas were evaluated for productivity (or competitive fitness) under three well-defined conditions, baseline conditions that mimic a natural solar day (BC, 5% CO₂ in air, 14:10 light dark cycle with zenith at noontime), hyperoxic conditions (HO, 5% CO₂ in 02), or light stress (LS, long periods of very low light) conditions.

For the LS and HO competition experiments, the pre-conditioning phase was reduced to a single day to avoid imposing long-term selection under the baseline conditions (BC). For the BC and hyperoxic conditions, standard illumination was provided on a 14:10 hour (light:dark) diurnal cycle simulating a cloudless day, with light intensity ascending to a zenith with maximum photosynthetically active radiation (PAR) of about 2000 μmol photons per square meter per second, and descending until dark, delivered in a sinusoidal form, as illustrated in the inset to FIG. 1C. For the LS regime, the standard illumination days were alternated with a series of three “light starvation” days, which consisted of a simple, 14:10 hour rectangular wave with a PAR intensity of 50 μmol photons per square meter per second. All cultures were stirred at 200 rpm using a 28.6 mm by 8 mm Teflon coated stir bar. Gas for BC and LS conditions was 5% CO₂ in air and gas for hyperoxic was 5% CO₂ in 02. Gas delivered through a 5 mm gas dispersion stone with a porosity of 10-20 microns at a flow rate of 250 ml/min for 60 seconds every hour. Culture temperatures were maintained at room temperature (RT) for the F1 and F2 competition and 25° C. for monoculture phenotyping of parental lines and competition survivors.

### Biomass Productivity

Biomass productivity was determined by multiplying the number of daily turbidistat dilutions (5 ml per dilution) and the Ash free dry weight (AFDW) then dividing by the area of the top of the ePBR culture vessel at 15 cm (0.002687 m²). Ash free dry weight was determined by concentrating 35 ml of culture onto a Whatman CF/F glass filter and dried over night at 104° ° C. prior to weighing for the “dry weight.” Organic matter was removed from the filter by heating the samples to 550° C. for a minimum of 30 minutes prior to weighing the sample for the “ash weight.” The AFDW is the dry weight minus the ash weight.

### Deep Sequencing

DNA samples of the pooled F1 progeny used as the inoculum for both population studies and samples from 2x-BC and 3x-hyperoxic populations on days 9, 21, 25, and 32 as well as 3x-BC and 3x-LR populations on days 6, 12, and 19 was isolated from the cells as described in Fawley & Fawley (2004). Genomic DNA library generation for was performed by the Michigan State University Genomics Core Facility using the Illumina TruSeq Nano DNA Library (see website at www.illumina.com) with dual 8 bp index adapters. Libraries were checked for quality and quantified using Qubit dsDNA HS, Caliper LabChipGX HS DNA (see website at www.perkinelmer.com) and Kapa Biosystems Illumina Quantification qPCR assays (www.perkinelmer.com). Libraries pooled for multiplexed sequencing and loaded on 2 lanes of an Illumina HiSeq 2500 High Output flow cell (v4) and sequencing was performed with HiSeq SBS reagents (v4) in a 2×125 bp paired end format. Base calling was done by llumina Real Time Analysis (RTA) v1.18.64 and output of RTA was demultiplexed and converted to FastQ format with Illumina Bcl2fastq v1.8.4. This generated an average of 6.05 Gb of sequence data per sample which came out to about 47×genomic coverage per sample for the F1 progeny competition. Libraries for the tetrad analysis and F2 competition were prepared using the Illumina TruSeq Nano DNA Library Preparation Kit on a Perkin Elmer Sciclone G3 robot following manufacturer's recommendations. Completed libraries were quality controlled and quantified using a combination of Qubit dsDNA HS and Caliper LabChipGX HS DNA assays. All libraries were combined in equimolar amounts and the pool quantified using the Kapa Biosystems Illumina Library Quantification qPCR kit. This pool was loaded onto 2 lanes of an Illumina HiSeq 4000 flow cell and sequencing performed in a 2×150 bp paired end format using HiSeq 4000 SBS reagents. Base calling was done by Illumina Real Time Analysis (RTA) v2.7.6 and output of RTA was demultiplexed and converted to FastQ format with Illumina Bcl2fastq v2.19.0. The average genomic sequencing depth of the tetrad and F2 experiments was ˜32×. Genomic DNA read pairs were aligned the Chlamydomonas reference genome v5.0 (JGI v5.0 assembly, JGI annotation based on Augustus u11.6) using the bowtie2/2.2.3 aligner. For each sam file output, the file was converted to bam and the reads were sorted, bam file head group fixed, mate information was fixed, and duplicated mates were removed using picardTools/1.113 (see website at github.com/broadinstitute/picard/). Reads were realigned to the reference genome using GATK3.1.1 (McKenna et al., 2010). Variant base calls were identified using SamTools/0.0.19 (Li et al., 2009) and output was filtered and formatted into the variant call format using vcftools/0.1.12a (Danecek et al., 2011).

### Allele Frequency Determination and Identification of Enriched Genomic Loci (EGLs)

For the progeny competition, CC1009 and CC2343 allele frequencies within the populations were determined by parsing and filtering the variant call output for our singleton SNP list, a gift from Jonathan Flowers described in Flowers et al. (2015). From the VCFTools output we used the quantified read data for all mapped SNPs unique to either CR1009 or CR2343 to determine the SNP frequency (SNP reads/SNP reads+Reference reads) for each singleton SNP. The SNP frequencies for both parents were then merged after inverting the reference and SNP read frequencies for CC1009 SNPs, thus orienting all SNP frequencies to the CC2343 parental line. Final allele frequencies reported here were determined by adjacent averaging all SNP frequencies using 40 Kb windows and repeating the window every 8 Kb down each chromosome. To determine regions of the genome with significant changes in SNP frequency for each environmental condition (BC, HO or LS) and assay time-point, we estimated the frequencies of markers attributable to CC2343, using a running average across 10 Kb windows centered every 8 Kb for each chromosome as described above. The statistical significance of differences between any pair of samples was calculated. Enriched genomic loci (EGLs) were defined as a region of the genome whose average p-value for difference in CR2343 frequency showed p<10-14. Enriched genomic loci (EGLs) were selected that were ≥60 kb in size.

### Refined Single Nucleotide Polymorphisms (SNPs).

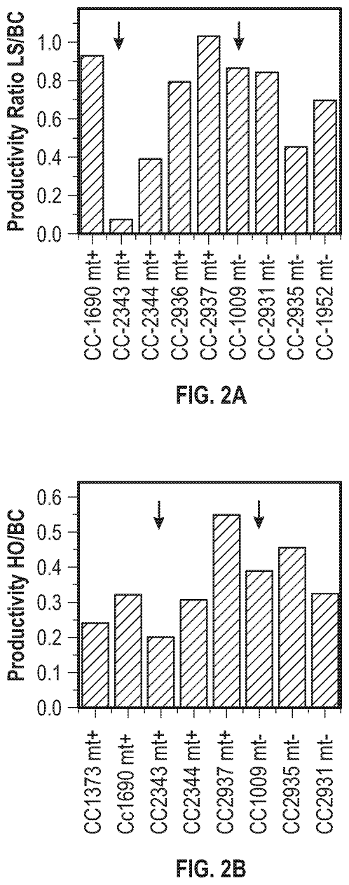

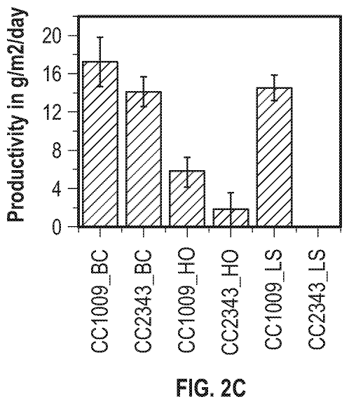

To map the parental allele frequency in polyculture populations of CC2343 and CC1009 meiotic progeny a list of single nucleotide polymorphisms (SNPs) was obtained from the parental strains. The approximate 2.6 million SNPs between CC2343 and CC1009 relative to the sequenced CC503 strain (Flowers et al., 2015) were initially employed. The list was then refined to sites that could be used quantitatively between the two parental lines. The genomes of CC2343 and CC1009 were re-sequenced and the reads were pooled into three sets of 24 million reads containing either 75%, 50%, or 25% of CC2343 and CC1009. After aligning the computational population, about 1 million SNPs that deviated more than 15% from the target frequency were removed from the Flowers list, resulting in over 1.6 million SNPs assigned to CC2343 or CC1009. A population of 203 mt+F1 progeny of CC2343 and CC1009 were generated, the population was pooled into equal numbers to use as inoculums for environmental competition experiments (FIG. 2A).

### Example 2: Light Stress and Hyperoxic Conditions Reduce Productivity of Chlamydomonas CC1009 and CC2343 Cultures

A series of natural isolates and progenitors to laboratory strains of Chlamydomonas were screened for productivity (or competitive fitness) under three well-defined conditions, baseline conditions that mimic a natural solar day (BC, 5% CO₂ in air, 14:10 light dark cycle with zenith at noontime), hyperoxic conditions (HO, 5% CO₂ in 02), or light stress conditions (LS, long periods of very low light) as described in Example 1 (see also FIG. 1A-1B).

One pair of lines, CC1009 and CC2343, exhibited similar growth under baseline conditions, but strong phenotypic differences under both hyperoxic and light stress conditions (FIG. 2A-2C). Growth experiments on monocultures (FIG. 2C) showed that CC1009, a mt− strain originally isolated from Massachusetts as highlighted by (Proschold et al., 2005), exhibited higher survival or fitness under both hyperoxic conditions and light stress conditions compared to CC2343, a mt+ ecotype isolated from Melbourne, Fla. (Spanier et al., 1992). Compared to baseline conditions, placing cultures under light stress conditions resulted in small (about 20%) losses in productivity of CC1009, but complete inhibition of growth of CC2343. A similar trend was found that under hyperoxic conditions, where both strains had reduced productivity, but CC1009 had a 66% decrease in productivity whereas CC2343 lost about 87%.

### Example 3: Allele Frequency Tracking by SNP Mapping of Mixed Chlamydomonas CC1009 and CC2343 Populations

The allele frequency of mixed populations of Chlamydomonas CC1009 or CC2343 strains that were generated as described in Example 2, were evaluated by single nucleotide polymorphism analysis using a refined list of single nucleotide polymorphisms (SNPs, see Example 1) from the parental strains for comparison.

The similar allele frequency (AF) distributions for the F1 inoculums show that population pooling, deep sequencing and SNP tracking methods generated highly reproducible results. Excluding chromosome 6 (CHR6), the CC2343 allele frequency varied between 0.5 and 0.35 across the genome. Allele frequencies of 0.42 and 0.58 for CC2343 and CC1009 respectively, were obtained after averaging all allele frequencies across the genome, indicating a slight bias for CC1009 within the population. By contrast, the 700-kb segment of DNA at the beginning of CHR6, corresponding to the mating type locus (MTL) (Ferris et al., 1994, 2002; De Hoff et al., 2013), showed strong selection for CC1009 loci. This served as a positive control for the methods described herein because mt+ strains were exclusively selected for the F1 competition experiments and the AF of the MTL within the population was, as expected, essentially homozygous for the CC1009 (mt+) parental locus, (FIG. 1C, blue shaded area).

The preference for CC1009 loci was progressively lost moving away from the MTL locus, indicating that crossover events must have occurred following mating. The largest changes in allele frequency occurred in two distinct regions of CHR6, together totaling less than 1 MB (FIG. 1C, grey shaded regions), suggesting regions of relatively high cross-over frequency, i.e. potential recombination “hotspots.”

### Example 4: Stress Conditions Induce Selection of Genomic Loci

The pooled F1 progeny libraries described in Examples 2 and 3 were cultured in ePBRs and grown under baseline conditions that mimic a natural solar day (BC, 5% CO₂ in air, 14:10 light dark cycle with zenith at noontime), hyperoxic conditions (HO, 5% CO₂ in 02), or light stress (LS. long periods of very low light) conditions (see Example 1 and FIG. 1A). Triplicate reactors for baseline conditions and triplicate reactors for either hyperoxic or light stress conditions were inoculated with each pooled population.

To follow the dynamics of selective enrichment for genetic loci, HO, LS and corresponding BC samples were collected for DNA isolation and subsequent deep sequencing.

A summary of samples collected, and their sequence coverages is provided in Table 1.

TABLE 1
Summary of the genome coverage
for each deep sequencing sample

| | Sample | | | | |
|---|---|---|---|---|---|
| Experiment | F1 inoculum 1 | | | | |
| F1 HO | (42) | Day 9 | Day 21 | Day 25 | Day 31 |
| | BC1 | 40 | 38 | 42 | 42 |
| | BC2 | | | | |
| | BC3 | 41 | 42 | 43 | 44 |
| | HO1 | 41 | 43 | 42 | 43 |
| | HO2 | 44 | 41 | 40 | 43 |
| | HO3 | 42 | 42 | 43 | 56 |

| | F1 inoculum 2 | | | |
|---|---|---|---|---|
| F1 LS | (55) | Day 6 | Day 12 | Day 19 |
| | BC1 | 60 | 56 | 54 |
| | BC2 | 51 | 52 | 50 |
| | BC3 | 47 | 52 | 47 |
| | LS1 | 58 | 53 | 58 |
| | LS2 | 54 | 51 | 51 |
| | LS3 | 54 | 53 | 49 |
| | F2 inoculum | | | |
| F2 | (38) | Day 8 | Day 16 | Day 21 |
| | BC1 | 36 | 29 | 35 |
| | BC2 | 31 | 29 | 34 |
| | BC3 | 32 | 27 | 32 |
| | HO1 | 33 | 36 | 41 |
| | HO2 | 39 | 35 | 28 |
| | HO3 | 38 | 42 | 35 |
| | LS1 | 28 | 32 | |
| | LS2 | 32 | 33 | |
| | LS3 | 34 | 35 | |

| | |
|---|---|
| Tetrad | F1_1_1 (51) |
| | F1_1_2 (38) |
| | F1_1_3 (109) |
| | F1_1_4 (52) |
| | F1_5_1 (56) |
| | F1_5_2 (35) |
| | F1_5_3 (41) |
| | F1_5_4 (92) |
| Parental | CC1009 (44) |
| | CC2343 (62) |

Biological replicates gave very similar patterns and extents of allele frequencies, indicating that the environmental conditions produced reproducible selections.

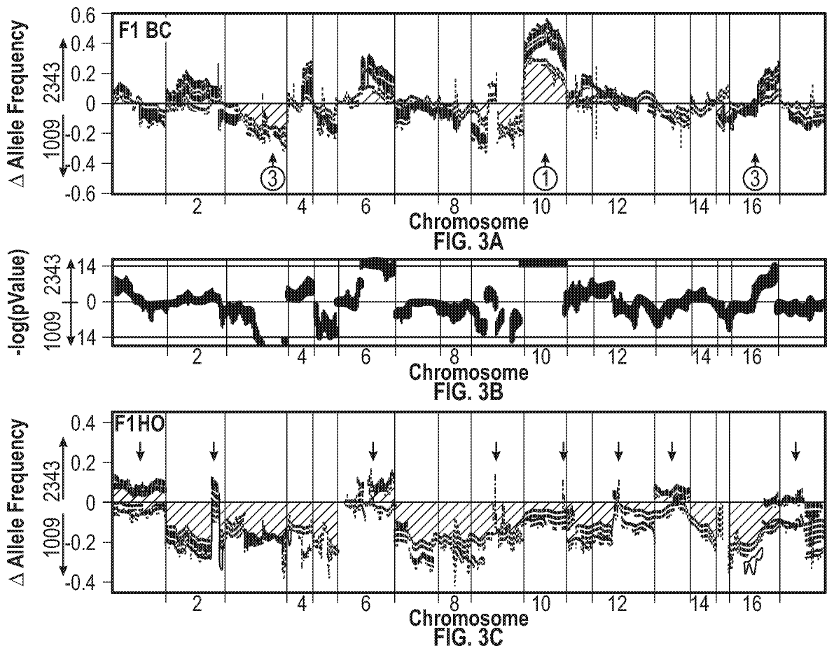

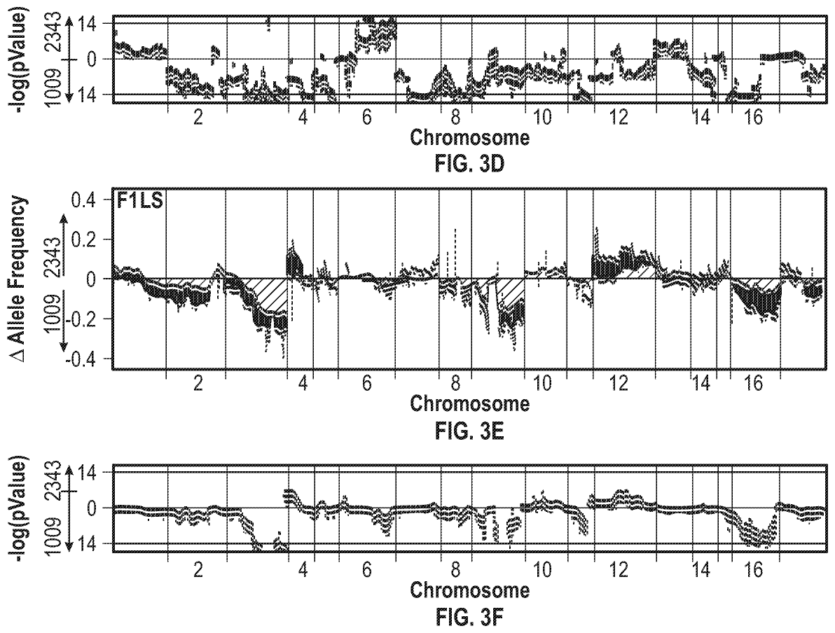

Typical QTL mapping measures the correlation between observed phenotypes and the occurrence of genetic markers in a set diversity panel. By contrast, the methods described herein quantify the enrichment or depletion of genomic loci in a pooled diversity panel after environmental selection. The resulting enrichment of loci is related to the fitness imposed by a loci or combination thereof. Because the statistical analyses and the implications of the approaches are distinct, the term Enriched Genomic Loci (EGL, pronounced eagle) was introduced specifically to indicate genomic regions that are significantly enriched (FIGS. 3B, 3D and 3F).

Each of the environmental conditions tested gave rise to distinct patterns of AF changes and Enriched Genomic Loci (FIG. 1), indicating that the environmental conditions imposed qualitatively different selection pressures for specific subsets of loci. Even though the baseline conditions were designed to be relative non-selective, it imposed rapid differential selection for specific loci, including “alternating banding” for selection from both parents on chromosomes 1, 9, and 16, and particularly strong selection for CC2343 alleles on chromosome 10 with the peak near the centromere (FIG. 3A). However, the average contribution of genomic loci from the two parents remained similar throughout the baseline condition competition, with only a slight (˜1.8%) preference for increases from CC2343 compared to the initial inoculums, consistent with the fact that the parent lines grew at nearly the same rate under these conditions.

In contrast to the baseline conditions, the more stressful hyperoxic and light stress conditions favored enrichment of CC1009 over CC2343, by 15% and 3.0% for hyperoxic conditions and light stress conditions respectively, in the final populations (Table 2), likely reflecting the higher tolerance and productivity of CC1009 under these conditions.

TABLE 2
The Percent Change of the Allele Frequency from the Initial Inoculum to CC2343

| | Condition | Day 9 | Day 21 | Day 25 | Day 31 |
|---|---|---|---|---|---|
| F1 HO | BC | 0.001 | 0.006 ± 0.020 | −0.006 ± 0.014 | −0.018 ± 0.006 |
| | HO | 0.132 ± 0.002 | 0.121 ± 0.017 | 0.112 ± 0.033 | 0.150 ± 0.049 |

| | | Day 6 | Day 12 | Day 19 |
|---|---|---|---|---|
| F1 LS | BC | 0.010 ± 0.031 | 0.006 ± 0.009 | −0.028 ± 0.006 |
| | LS | 0.021 ± 4.85e−4 | 0.043 ± 0.005 | 0.030 ± 0.005 |
| | | Day 8 | Day 16 | Day 21 |
| F2 All | BC | 1.76e−4 ± 0.005 | −0.032 ± 0.008 | −0.046 ± 0.005 |
| | O2 | −0.001 ± 0.007 | −0.011 ± 0.003 | 0.004 ± 0.003 |
| | LR | 0.022 ± 0.004 | 0.023 ± 0.001 | |

It is noteworthy that all the final populations contained combinations of loci from both parents, though in distinct patterns. For example, hyperoxic conditions resulted in:

a) selection for long stretches of CC1009 alleles throughout the genome, that were interspersed with short blocks from CC2343, especially on chromosomes 1, 2, 6 and 13;

b) relatively low selectivity on first 25% of chromosome 4, heavily heavy selection for CC1009 alleles on the latter 75%;

c) selection for CC1009 on most of chromosomes 10 and 12.

By contract, light stress conditions resulted in:

a) enrichment of CC2343 loci on the first half of chromosome 4 but a slight preference for CC1009 loci on the second half;

b) enrichment for CC2343 alleles on chromosomes 10 and 12; and

c) bi-parental inheritance for segments from both parents on the first −3.5 Mb of chromosomes 17, but preferential selection for CC2343 on the latter while the right 3.5 Mb showed selection for CC1009.

Taken together, these diverse responses indicate that each environmental condition selects for distinct combinations of loci, and that those distinct can be linked to increased fitness under the correlated environmental condition.

The time-dependence of allele selection for individual genomic regions also followed different kinetic patterns. For most regions, the largest allele frequency changes appeared during the first 6-9 days after inoculation, followed by smaller adjustments in the later time points (FIG. 3A, arrow 1). However, some regions, including chromosome 3, showed strong immediate selection followed by little change throughout the experiment (FIG. 3A, arrow 2), while some loci showed a relatively steady rate of change (FIG. 3A, arrow 3). These differences indicate the importance of the most impactful loci first, followed by slower selection for secondary effects of various combinations of loci.

In other cases, initial rapid selection for loci from one parent was slowly reversed (FIG. 3C, arrows). This could be selection for loci encoding important gene networks, or selection for loci that enable rapid adaptation to the environment followed by loci that eventually acclimate to hyperoxic conditions. Under light stress, the overall rate of changes was slower than under baseline or hyperoxic conditions, which may be due to the low numbers of cell divisions during the low light days.

### Example 5: Mating-Induced Genomic Diversity

This Example describes mating-induced genomic diversity occurs following F1 crosses and illustrates the selective differences and enriched genomic loci (EGLs) mapping resolution of such mating-induced genomic diversity.

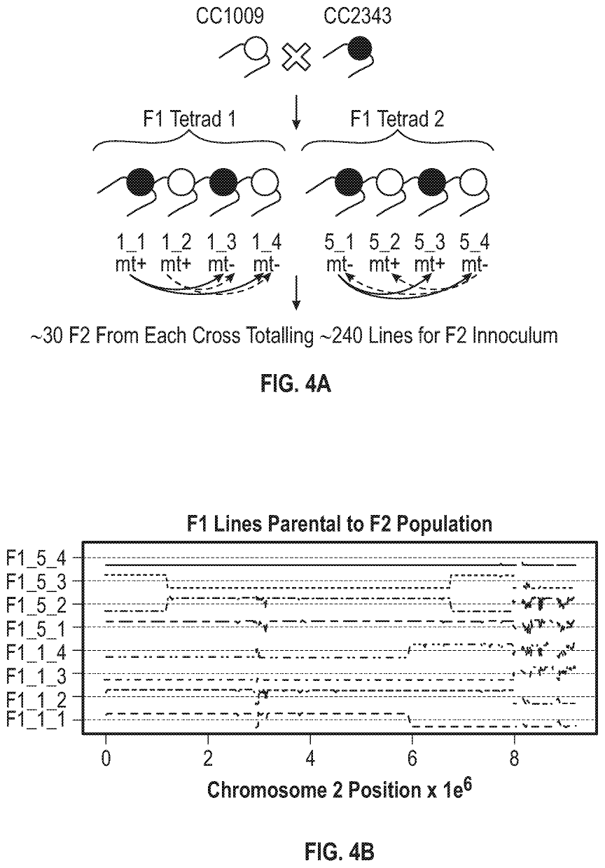

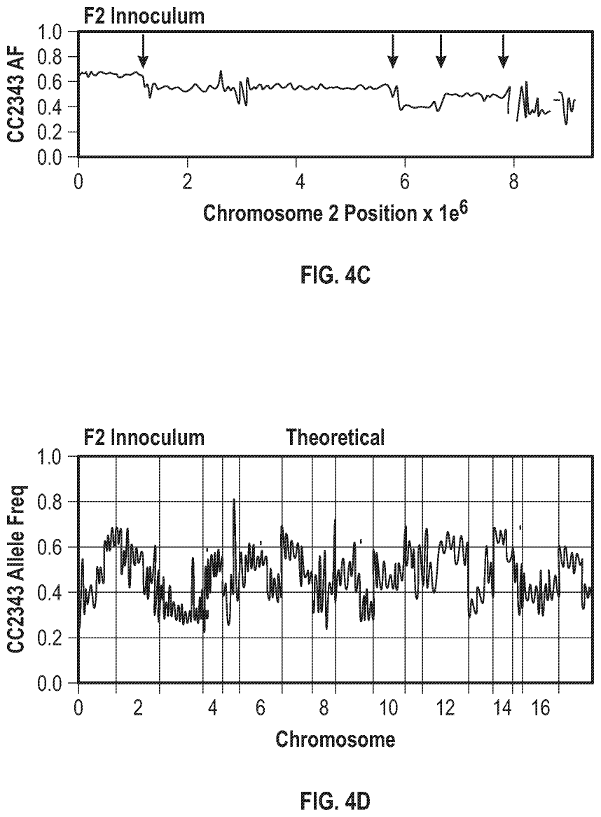

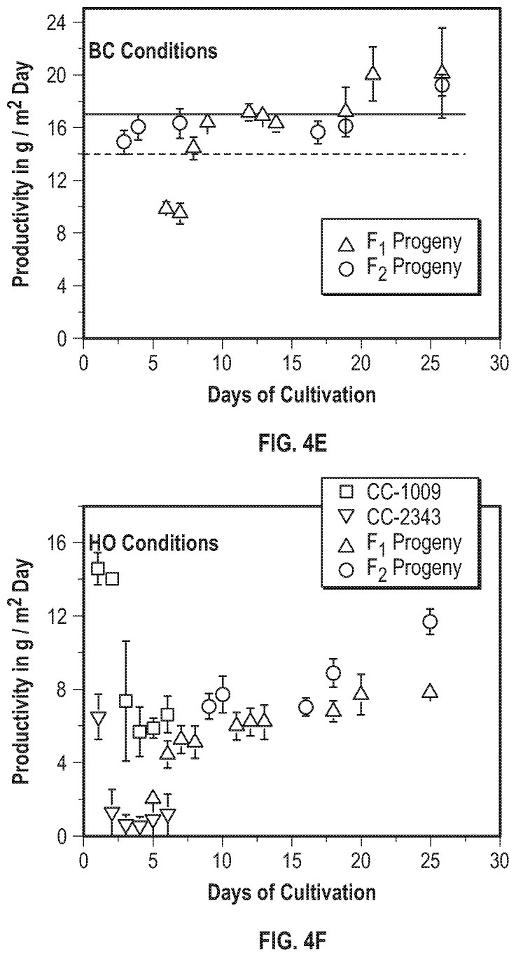

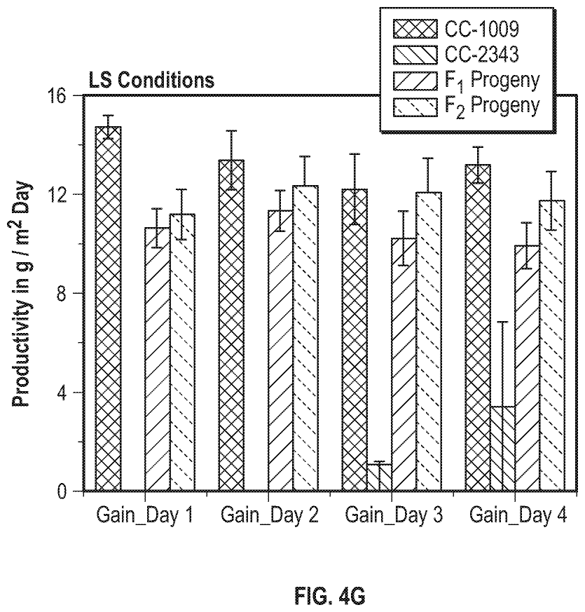

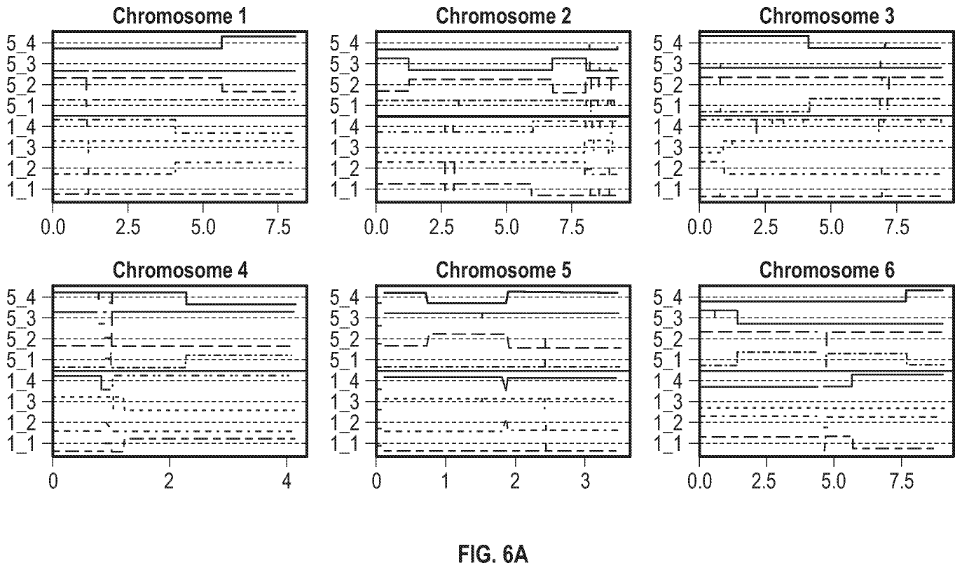

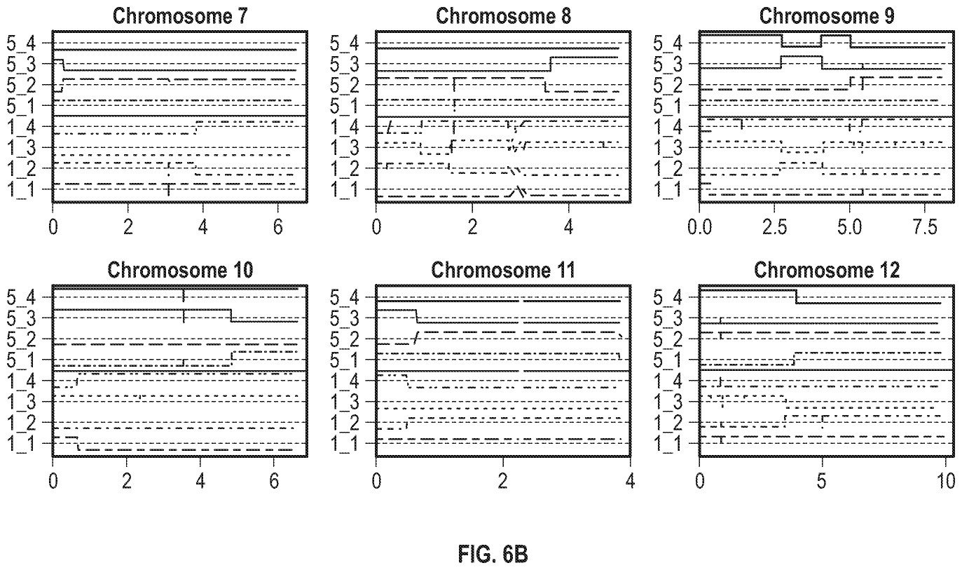

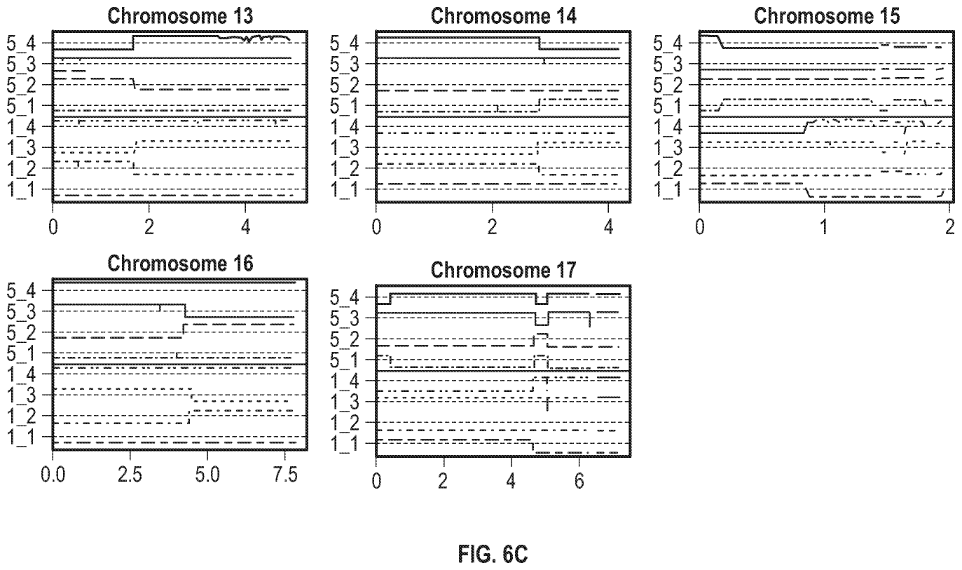

To better understand the homologous recombination in Chlamydomonas, and its effects on the genomic structure and selective advantage, an F2 population was generated by intercrossing F1 progeny prior to selection (FIG. 4). The progeny from two dissected F1 tetrads, were sequenced and the genomic loci corresponding to CC1009 or CC2343 was mapped (FIG. 4B; FIG. 6).

The F1 progeny showed an average of about 13 crossover events for each cell, distributed over the 17 chromosomes (see examples in FIG. 1C), providing a rough baseline for the rate of genetic diversification during meiosis in Chlamydomonas. The mt- and mt+ individuals from these tetrads were then intercrossed to generate an F2 population, which was pooled and deep sequenced. The distribution of loci from each parent deviated substantially from the theoretical expectation of equal contributions from each parent (FIG. 4D), with nearly all of chromosomes 3 and 16, and significant portions of chromosomes 5, 9, 13 and 16 showing enrichment of loci from CC1009 between 10 and 17% (FIG. 4D), indicating that the second mating itself may have imposed selection for certain genomic loci.

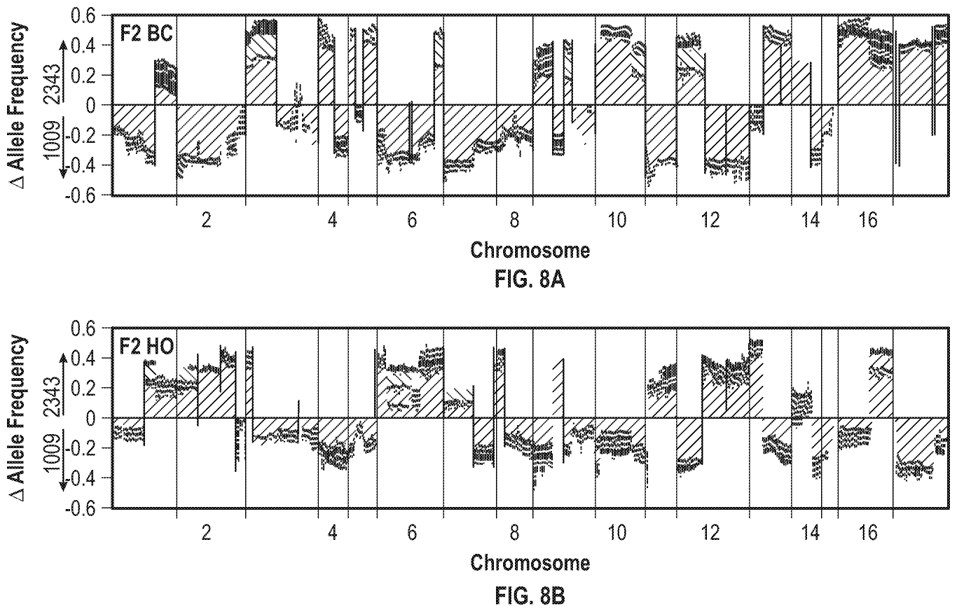

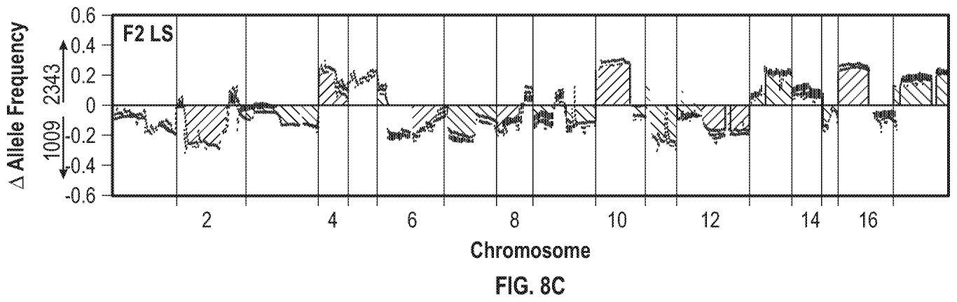

The pooled F2 progeny were incubated under baseline conditions, hyperoxic conditions, or light stress conditions. Samples were collected at days 8, 16 and 21 for deep sequencing to track the allele frequency of each population (Tables 1 and 2; FIG. 8).

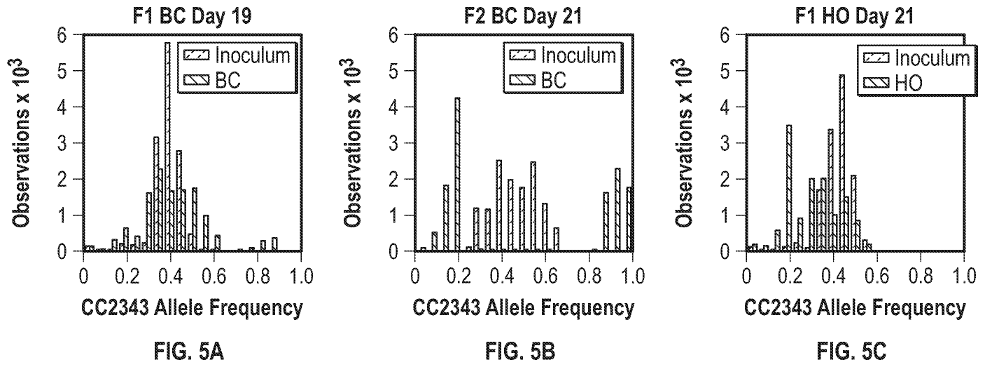

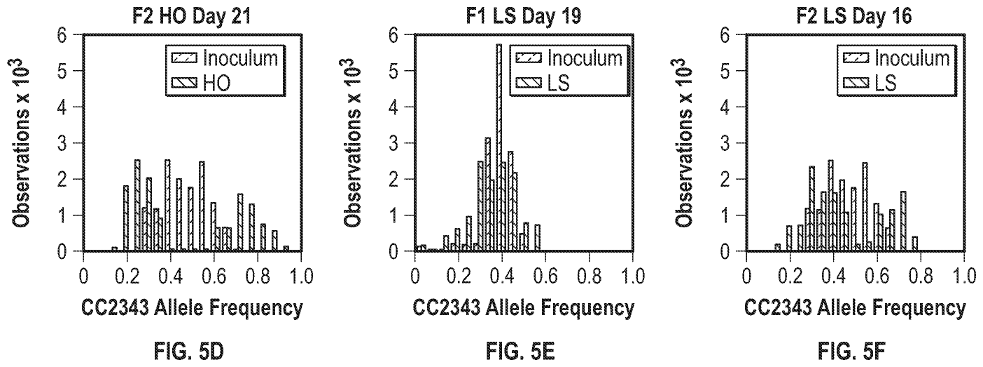

As with the F1 pool, competing the F2 under different environmental conditions led to enrichment of distinct combinations of genomic regions, but with some important differences. The F1 competitions resulted in nearly Gaussian distributions of allele enrichments (FIG. 5), indicating that the final pool contained a range of genetic variants that could compete relatively evenly.

By contrast, the F2 competition, particularly under baseline conditions, imposed nearly complete selection for regions from one progenitor or the other (FIG. 4). In some cases, the extreme selection for one set of alleles made accurate enriched genomic loci mapping of the F2 population difficult, because accurate mapping requires mapping of alleles from both parents to a reference genome. The resulting strongly bimodal enrichment distributions (FIG. 6: FIG. 8) were consistent with lower genome diversity. The results indicate that a smaller number of progeny can outcompete the others, i.e. the competitive advantage for individuals in the F2 populations was likely dominated by a relatively small number of key genomic regions, leading to strong founder effects. This conclusion is supported by F2 populations after incubation under selection conditions, where these populations retained stretches of the chromosome that contains the crossover positions from the individual dissected F1 tetrads (FIG. 6), indicating that relatively few additional alterations occurred, and this can partly be explained if a fraction of the secondary crossover events were silent.

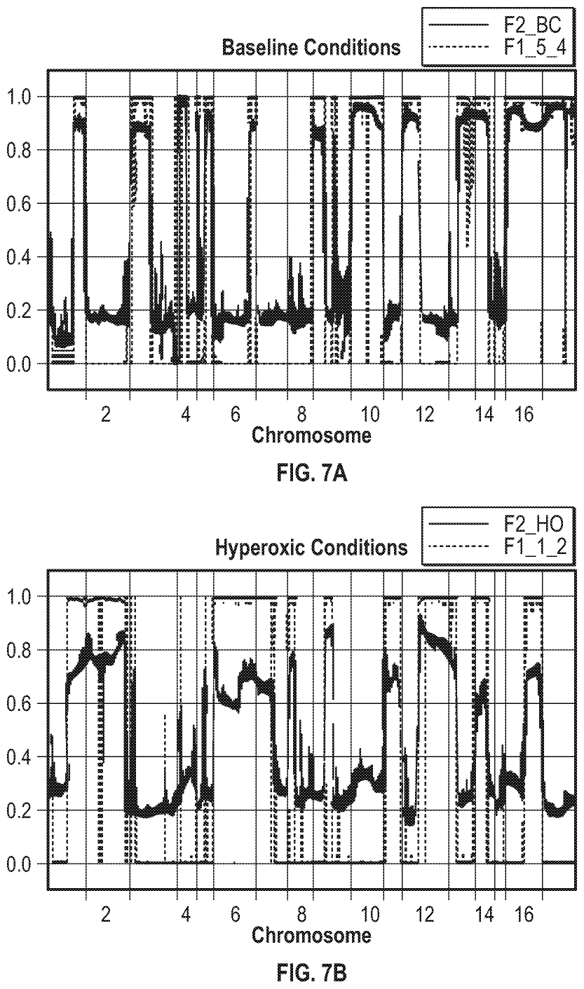

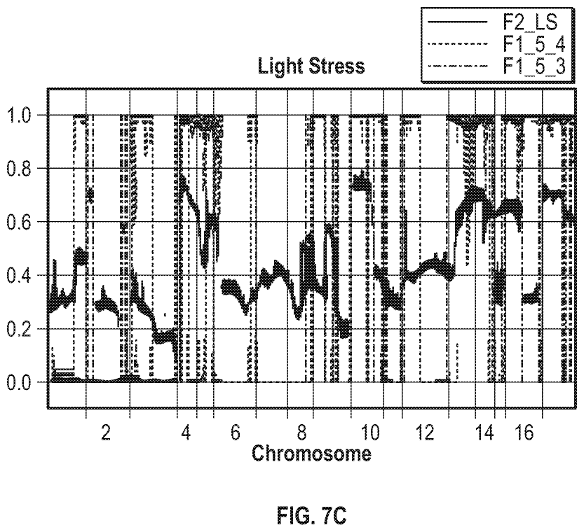

Interestingly, each environmental condition selected for different genomic regions from the tetrad parents. For example, baseline conditions selected almost exclusively for genomic regions with crossovers that matched a single F1 tetrad (termed F1_5_4, see FIG. 7A). To a lesser extent hyperoxic conditions selected for loci from a different tetrad parent (F1_1_2) as well as diversity from crossovers that were not seen in the dissected tetrads and thus likely arose from meiosis during F2 mating (see. e.g. see arrows in FIG. 7B indicating abrupt changes in allelic frequency in chromosomes 1, 3, 9 and 13). Light stress produced a population with the highest genomic diversity, as shown by the more Gaussian distributions of allelic frequency (FIG. 5C), and clear contributions from at least two tetrad parents (F1_5_3 and F1_5_4) (see arrows in FIG. 7C).

### Example 6: Chlamydomonas Shows Strong Heterosis (“Hybrid Vigor”)

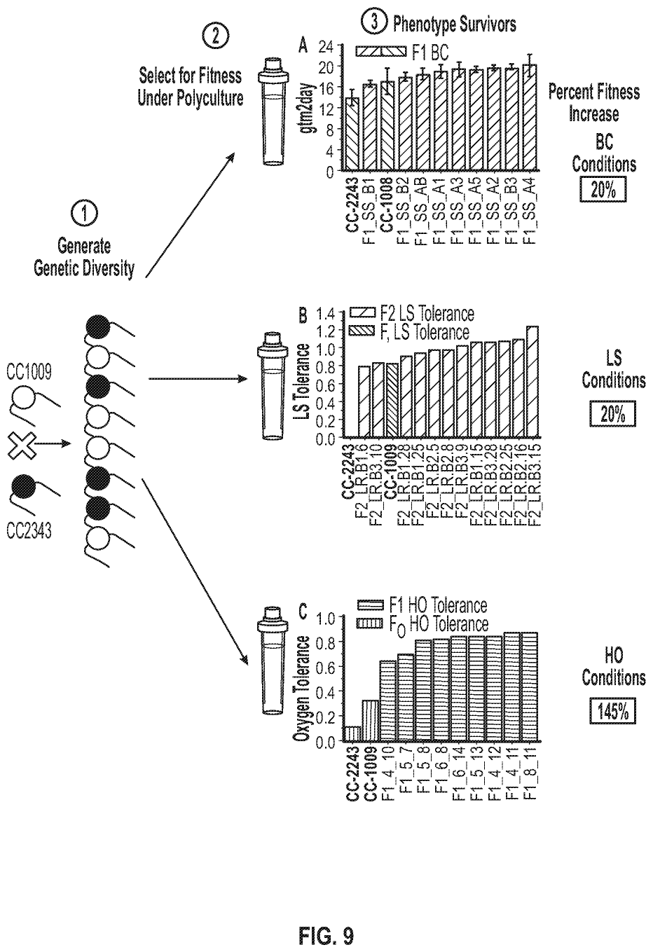

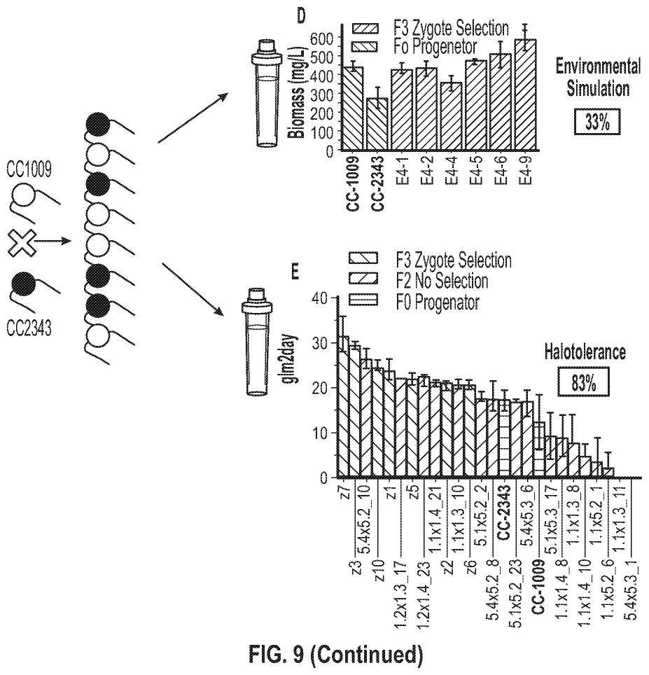

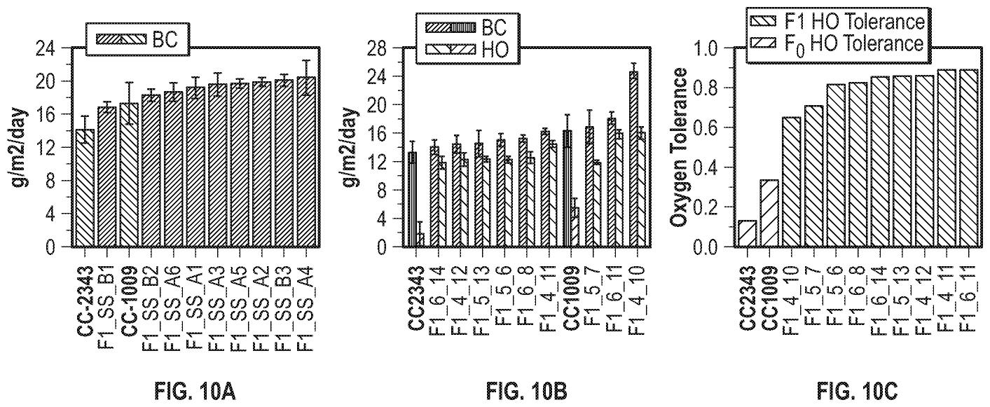

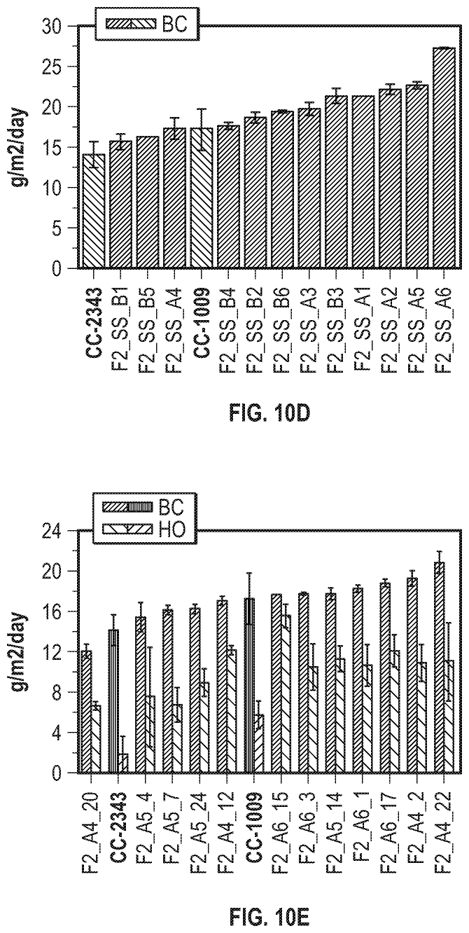

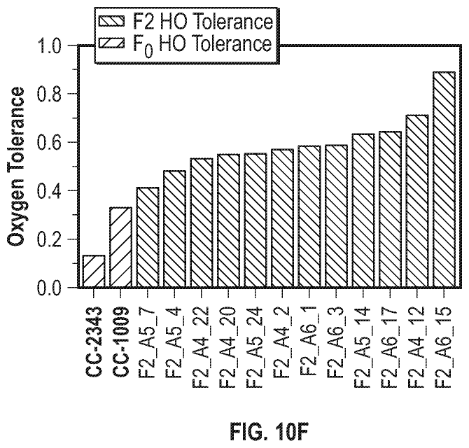

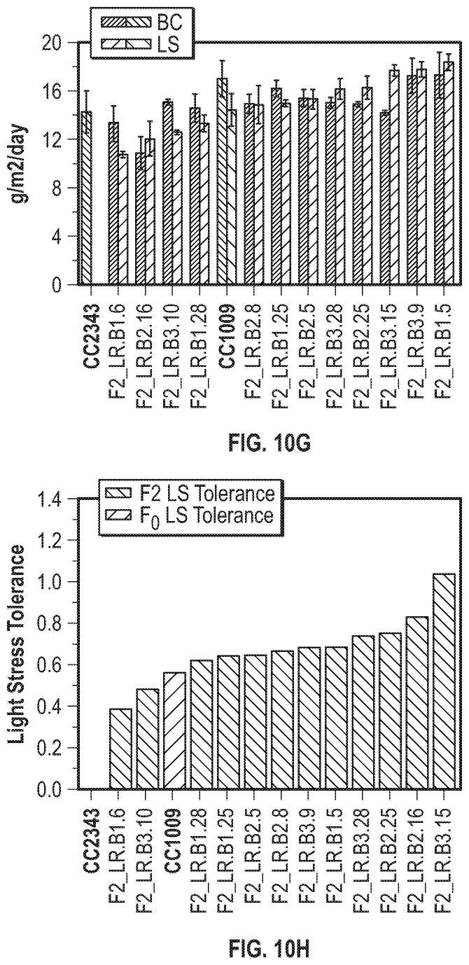

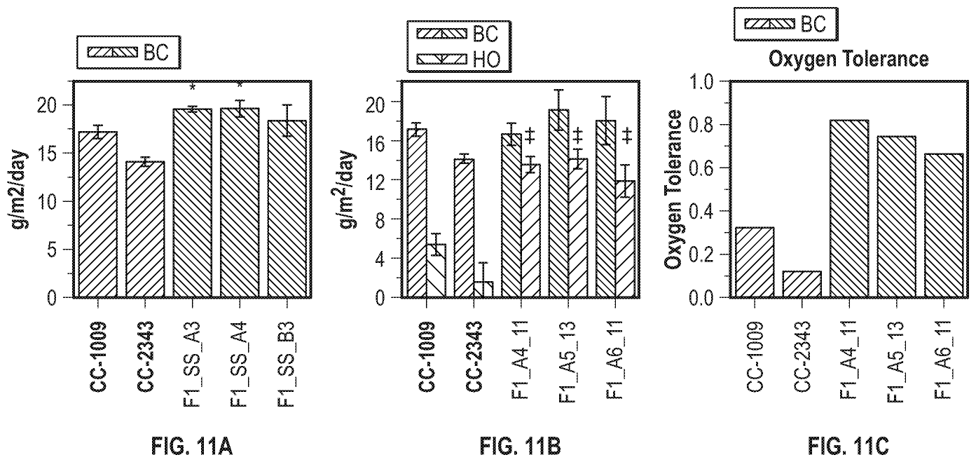

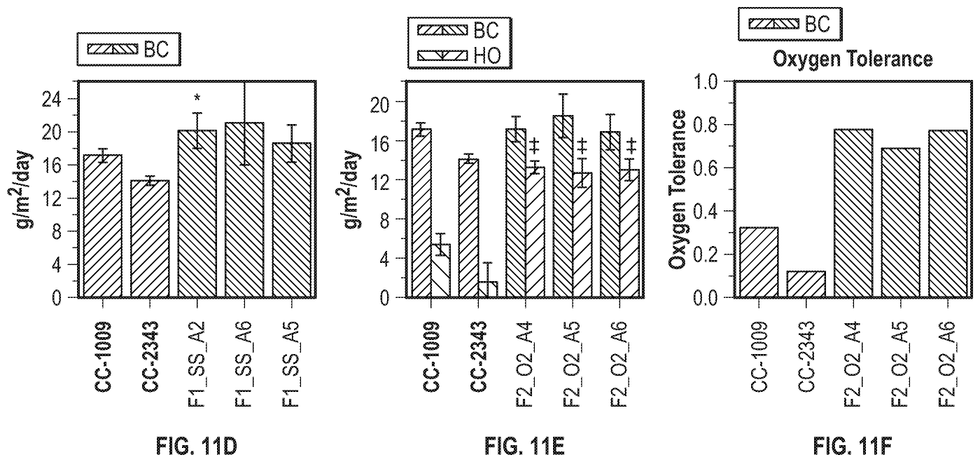

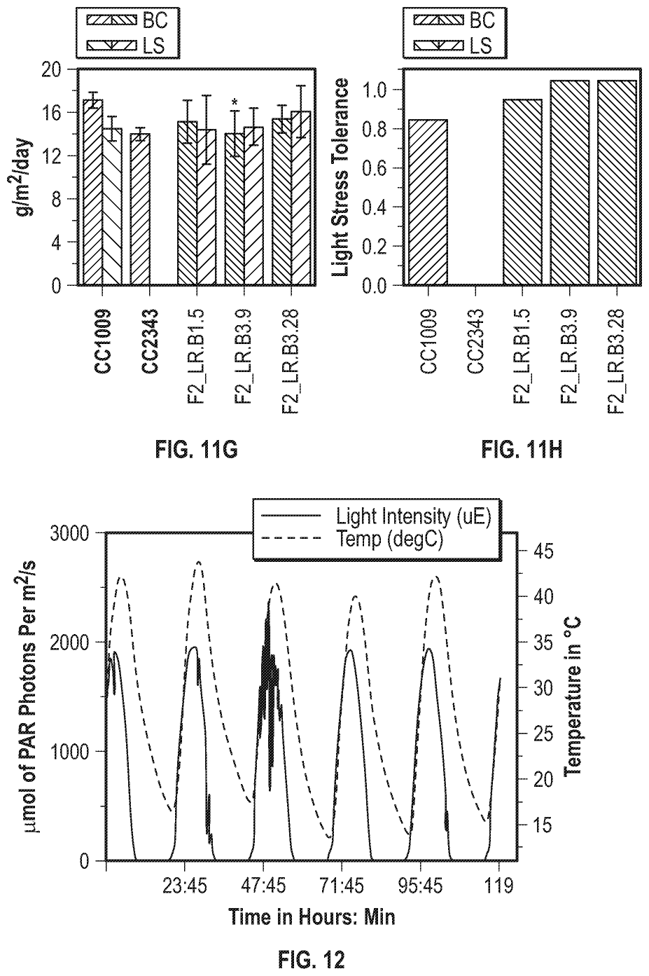

The maximal and cumulative productivities of the pooled F1 and F2 polycultures under baseline or hyperoxic conditions surpassed that of either of the parental lines, suggesting that the increased genetic diversity led to heterosis (FIG. 9). Thus 9-12 “winners” were isolated from the final F1 and F2 competition cultures and compared their productivities under their respective selection conditions (FIGS. 10-11). Strikingly, the majority of winners from both F1 and F2 populations displayed productivity or tolerance that exceeded that of either of the original parent lines (FIG. 9). When the best performing winners from the F1 competition under baseline conditions were subjected to hyperoxic conditions, the survivors showed 20% and 145% increases in biomass productivities compared to the best performing of the parent lines. Similar trends were observed with F2 winners, though the extent of improvement varied (see FIGS. 10-11). The extended performance of the winners implies that mating led to heterosis. Compared to CC1009, hyperoxic survivors exhibited similar or even more robust growth under baseline conditions (FIGS. 11B and 11E), whereas some light stress winners exhibited a decrease baseline condition productivity (FIG. 11H), but a higher ratio of light stress:baseline condition productivity, indicating that increased productivity under some conditions may be translated to others, but in some cases, might lead to tradeoffs in some phenotypic characteristics.

To further explore the genetic plasticity of Chlamydomonas, the mating and selection process was streamlined by hatching pools of hundreds to thousands of isolated zygotes, during, or just prior to, imposition of selection conditions relevant to algal production. In the first experiment zygotes were hatched prior to exposure of harsh conditions experienced in an algal growth pond (PoCo), with fluctuating of temperatures (between 12 and 44° C.) and high light (FIG. 12). As shown in FIG. 9D, all winners performed better than the poor performing parent line, CC2343, and one showed a statistically significant increase (˜33%) over the better performing parent, CC1009. In one experiment, zygotes were hatched under high salt conditions (HiSaCo, 20 g/L of Instant Ocean salts) and grown for grown for eight days. The growth of the progenitor lines, HiSa (high salt) survivors and 17 randomly selected F2 progeny was tested in HiSa media. The random F2 progeny displayed growth rates under HiSaCo ranging from zero (i.e. HiSaCo lethal) to well above that of the parent lines. However, all of the environmental selection winners showed strikingly higher productivity than either parental strains (FIG. 9E).

The foregoing results demonstrate that natural variants of Chlamydomonas contain genetic plasticity that, through the algal breeding and selection methods described herein, can generate algal lines with strong heterosis for growth and productivity under a wide range of environmental challenges. Quantitative genomics approaches can be used to identify EGL that reflect the genetic bases for the observed heterosis. The current resolution of the EGL regions identified from both F1 population spans 60 KB to over 1.2 MB, encoding from 10 to over 2000 genes, and thus far too low to identify specific genes linked to increased productivity. However, the results on both the F1 and F2 competitions indicate that increased enriched genomic loci (EGL) resolution could be obtained by generating massive libraries of primary and secondary crossover events, followed by generations of cross competition. Finally, in at least some cases, gains in productivity obtained by selection under one condition did not impose tradeoffs, or even led to modest increases in productivity, under other conditions, indicating that the methods described herein can be used to increase algal productivity under a range of conditions, especially for production environments.

### Example 7: Zygospore Hatching to Generate Populations

Zygospores may be generated by mating algae strains. The zygospores are then isolated and hatched to generate the diversity panel used for selection.

### REFERENCES

Anderson. J. T., Willis, J. H. and Mitchell-Olds. T. (2011) Evolutionary genetics of plant adaptation. Trends in Genetics, 27, 258-266.
Bertoni, G. (2015) Genomic Diversity in Chlamydomonas Laboratory and Field Strains. The Plant cell, 27, 2315-2316.
Craigie, R. A. and Cavalier-Smith, T. (1982) Cell Volume and the Control of the Chlamydomonas Cell Cycle. Journal of Cell Science, 54, 173.
Cross, F. R. and Umen, J. G. (2015) The Chlamydomonas cell cycle. The Plant Journal, 82, 370-392.
Danecek, P., Auton, A., Abecasis, G., Albers, C. A., Banks, E., DePristo, M. A., Handsaker, R. E., Lunter, G., Marth, G. T., Sherry, S. T., McVean, G., Durbin, R. and Genomes Project Analysis, G. (2011) The variant call format and VCFtools. Bioinformatics, 27, 2156-2158.
Davey, P. T., Hiscox, W. C., Lucker, B. F., O'Fallon, J. V., Chen, S. and Helms, G. L. (2012) Rapid triacylglyceride detection and quantification in live micro-algal cultures via liquid state 1H NMR. Algal Research, 1, 166-175.
De Hoff. P. L., Ferris, P., Olson, B. J., Miyagi. A., Geng, S. and Umen. J. G. (2013) Species and population level molecular profiling reveals cryptic recombination and emergent asymmetry in the dimorphic mating locus of C. reinhardtii. PLoS Genet, 9, e1003724.
Elena, S., Ekunwe, L., Hajela, N., Oden, S. and Lenski, R. (1998) Distribution of fitness effects caused by random insertion mutations in Escherichia coli. Genetica, 102-103. 349-358.
Ferris, P J. and Goodenough, U. W. (1994) The mating-type locus of Chlamydomonas reinhardtii contains highly rearranged DNA sequences. Cell, 76, 1135-1145.
Flowers, J. M., Hazzouri, K. M., Pham, G. M., Rosas. U., Bahmani, T., Khraiwesh. B., Nelson, D. R., Jijakli, K., Abdrabu. R., Harris, E. H., Lefebvre, P. A., Hom, E. F. Y., Salehi-Ashtiani, K. and Purugganan, M. D. (2015) Whole-Genome Resequencing Reveals Extensive Natural Variation in the Model Green Alga Chlamydomonas reinhardtii. The Plant Cell.
Hannah. M. A., Wiese. D., Freund, S., Fiehn, O., Heyer, A. G. and Hincha, D. K. (2006) Natural genetic variation of freezing tolerance in Arabidopsis. Plant physiology, 142, 98-112.
Hottes, A. K., Freddolino, P. L., Khare, A., Donnell, Z. N., Liu, J. C. and Tavazoie, S. (2013) Bacterial Adaptation through Loss of Function. PLoS Genet, 9, e1003617.
Jang, H. and Ehrenreich, I. M. (2012) Genome-Wide Characterization of Genetic Variation in the Unicellular. Green Alga Chlamydomonas reinhardtii. PloS one, 7, e41307.
Jiang, X. and Stern, D. (2009) Mating and Tetrad Separation of Chlamydomonas reinhardtii for Genetic Analysis. e1274.
Li, H., Handsaker, B., Wysoker, A., Fennell, T., Ruan, J., Homer, N., Marth, G., Abecasis, G., Durbin, R. and Genome Project Data Processing, S. (2009) The Sequence Alignment/Map format and SAMtools. Bioinformatics, 25. 2078-2079.
Lovell, J. T., Juenger, T. E., Michaels. S. D., Lasky, J. R., Platt, A., Richards, J. H., Yu, X., Easlon, H. M., Sen, S. and McKay, J. K. (2013) Pleiotropy of FRIGIDA enhances the potential for multivariate adaptation. Proceedings of the Royal Society B: Biological Sciences, 280.
Lucker. B. F., Hall. C. C., Zegarac, R. and Kramer, D. M. (2014) The environmental photobioreactor (ePBR): An algal culturing platform for simulating dynamic natural environments. Algal Research, 6, Part B, 242-249.
McKenna, A., Hanna, M., Banks, E., Sivachenko. A., Cibulskis, K., Kernytsky, A., Garimella, K., Altshuler, D., Gabriel, S., Daly, M. and DePristo, M. A. (2010) The Genome Analysis Toolkit: A MapReduce framework for analyzing next-generation DNA sequencing data. Genome Research, 20, 1297-1303.
McMinn, A., Pankowski, A. and Delfatti. T. (2005) EFFECT OF HYPEROXIA ON THE GROWTH AND PHOTOSYNTHESIS OF POLAR SEA ICE MICROALGAE 1. Journal of Phycology, 41. 732-741.
Norton, T. A., Melkonian. M. and Andersen, R. A. (1996) Algal biodiversity. Phycologia, 35, 308-326.
Pröschold, T., Harris, E. H. and Coleman. A. W. (2005) Portrait of a Species: Chlamydomonas reinhardtii. Genetics, 170, 1601-1610.
Raven, B. J. A., Johnston, A. M., Parsons, R. and KÜBler, J. (1994) THE INFLUENCE OF NATURAL AND EXPERIMENTAL HIGH 02 CONCENTRATIONS ON 02-EVOLVING PHOTOTROPHS. Biological Reviews, 69, 61-94.
Raven, J. A. and Larkum, A. W. (2007) Are there ecological implications for the proposed energetic restrictions on photosynthetic oxygen evolution at high oxygen concentrations? Photosynthesis research, 94, 31-42.
Rounsley, S. D. and Last, R. L. (2010) Shotguns and SNPs: how fast and cheap sequencing is revolutionizing plant biology. The Plant Journal, 61, 922-927.
Rymarquis, L. A., Handley. J. M., Thomas, M. and Stern, D. B. (2005) Beyond Complementation. Map-Based Cloning in Chlamydomonas reinhardtii. Plant physiology, 137, 557.
Spanier, J. G., Graham, J. E. and Jarvik, J. W. (1992) ISOLATION AND PRELIMINARY CHARACTERIZATION OF THREE CHLAMYDOMONAS STRAINS INTERFERTILE WITH CHLAMYDOMONAS REINHARDTII (CHLOROPHYTA)1. Journal of Phycology, 28, 822-828.
Sueoka, N. (1960) MITOTIC REPLICATION OF DEOXYRIBONUCLEIC ACID IN CHLAMYDOMONAS REINHARDI. Proceedings of the National Academy of Sciences of the United States of America, 46, 83-91.
Todesco, M., Balasubramanian, S., Hu, T. T., Traw, M. B., Horton, M., Epple, P., Kuhns, C., Sureshkumar, S., Schwartz, C., Lanz, C., Laitinen, R. A., Huang, Y., Chory, J., Lipka, V., Borevitz, J. O., Dangl, J. L., Bergelson, J., Nordborg, M. and Weigel, D. (2010) Natural allelic variation underlying a major fitness trade-off in Arabidopsis thaliana. Nature, 465, 632-636.
Wang, Y., Yang, L., Zheng. Z., Grumet. R., Loescher, W., Zhu, J.-K., Yang, P., Hu. Y. and Chan, Z. (2013) Transcriptomic and Physiological Variations of Three Arabidopsis Ecotypes in Response to Salt Stress. PloS one, 8, e69036.
Werner, J. D., Borevitz, J. O., Uhlenhaut, N. H., Ecker, J. R., Chory, J. and Weigel, D. (2005) FRIGIDA-Independent Variation in Flowering Time of Natural Arabidopsis thaliana Accessions. Genetics, 170, 1197-1207.

All patents and publications referenced or mentioned herein are indicative of the levels of skill of those skilled in the art to which the invention pertains, and each such referenced patent or publication is hereby specifically incorporated by reference to the same extent as if it had been incorporated by reference in its entirety individually or set forth herein in its entirety. Applicants reserve the right to physically incorporate into this specification any and all materials and information from any such cited patents or publications.

The following statements describe some of the elements or features of the invention. Because this application is a provisional application, these statements may become changed upon preparation and filing of a nonprovisional application. Such changes are not intended to affect the scope of equivalents according to the claims issuing from the nonprovisional application, if such changes occur. According to 35 U.S.C. § 111(b), claims are not required for a provisional application. Consequently, the statements of the invention cannot be interpreted to be claims pursuant to 35 U.S.C. § 112.

### Statements

1. A method for producing algae with strong hybrid vigor for photosynthetic productivity comprising
   (a) crossing (mating) phenotypically-diverse algae strains to generate two or more genetically diverse algae strains;
   (b) culturing (e.g. growing) one or more genetically diverse algae strain under one or more selection conditions to generate an environmentally competitive algae population;
   (c) measuring the photosynthetic efficiency and/or productivity of one or more algae strain of the an environmentally competitive algae population; and
   (d) isolating an environmentally competitive algae strain or a mixture of environmentally competitive algae strains that exhibit hybrid vigor under the selection conditions compared to at least one of the phenotypically-diverse algae strain(s) grown under baseline conditions, to thereby produce one or more environmentally competitive algae strain or a mixture of environmentally competitive algae strains that exhibit hybrid vigor.
2. The method of statement 1, wherein the baseline condition comprises 5% CO₂ in air, and a 14-hour light:10 dark cycle with zenith at noontime.
3. The method of statement 1 or 2, wherein the baseline condition comprises light intensity ascending to a zenith with maximum photosynthetically active radiation (PAR) of about 2000 μmol photons per square meter per second (m⁻²s⁻¹), and descending until dark, delivered in a sinusoidal form.
4. The method of statement 1, 2 or 3, wherein the selection conditions comprise an increased oxygen atmosphere, a reduced carbon dioxide atmosphere, reduced light conditions, increased light conditions, increased salt conditions, increased temperatures, decreased temperatures, fluctuating temperatures, reduced nitrogen conditions, reduced pH conditions, increased pH conditions, conditions comprising macronutrients, conditions comprising micronutrients, conditions comprising pollutants, reduced phosphate conditions, or increased phosphate conditions.
5. The method of statement 1-3 or 4, wherein one of the selection conditions comprises hyperoxic atmospheric conditions comprising 5% CO₂ in oxygen.
6. The method of statement 1-4 or 5, wherein one of the selection conditions comprises reduced carbon dioxide atmospheric conditions comprising an atmosphere of less than 0.04% CO₂.
7. The method of statement 1-5 or 6, wherein one of the selection conditions comprises reduced light stress conditions comprising cycles of 1-3 days of baseline light followed by 1-3 days of very low light.
8. The method of statement 1-6 or 7, wherein one of the selection conditions comprises reduced light stress conditions comprising:
   a. one day of a baseline condition comprising 5% CO₂ in air, and a 14-hour light:10 dark cycle, wherein light intensity ascends at noon to a zenith with maximum photosynthetically active radiation (PAR) of about 2000 μmol photons per square meter per second (m⁻²s⁻¹), and descending until dark, delivered in a sinusoidal form; and
   b. followed by three light starvation days, each light starvation day comprising a 14-hour:10-hour light:dark, where the light comprises a rectangular wave with a PAR intensity of 50 μmol photons per square meter per second (m⁻²s⁻¹).
9. The method of statement 1-7 or 8, wherein one of the selection conditions comprises increased light conditions comprising more than 2000 μmol photons per square meter per second (m⁻²s⁻¹).
10. The method of statement 1-8 or 9, wherein one of the selection conditions comprises increased salt conditions comprising culturing the one or more genetically diverse algae strain in culture media comprising more than 0.2 M sodium chloride.
11. The method of statement 1-9 or 10, wherein one of the selection conditions comprises increased temperatures comprising culturing the one or more genetically diverse algae strain at more than 40° C.
12. The method of statement 1-9 or 10, wherein one of the selection conditions comprises decreased temperatures comprising culturing the one or more genetically diverse algae strain at less than 15° C.
13. The method of statement 1-11 or 12, wherein one of the selection conditions comprises fluctuating temperatures comprising culturing the one or more genetically diverse algae strain at fluctuating temperatures between 12° C. and 44° C.
14. The method of statement 1-12 or 13, wherein one of the selection conditions comprises reduced nitrogen conditions comprising culturing the one or more genetically diverse algae strain in culture media comprising less than 0.2 mM nitrate.
15. The method of statement 1-13 or 14, wherein one of the selection conditions comprises reduced phosphate conditions comprising culturing the one or more genetically diverse algae strain in culture media comprising less than 1 mM phosphate.
16. The method of statement 1-13 or 15, wherein one of the selection conditions comprises increased phosphate conditions comprising culturing the one or more genetically diverse algae strain in culture media comprising more than 2 mM phosphate.
17. The method of statement 1-15 or 16, wherein at least one of the phenotypically-diverse algae strain(s) is a species of Protococcus, Ulva, Codium, Enteromorpha, Neochloris and/or Chlamydomonas.
18. The method of statement 1-16 or 17, wherein at least one of the phenotypically-diverse algae strain(s) is a Chlamydomonas reinhardtii strain.
19. The method of statement 1-17 or 18, wherein measuring the photosynthetic efficiency and/or productivity of one or more algae strain of the an environmentally competitive algae population comprises measuring the number of daily dilutions (e.g. of 5 or 10 ml) needed to maintain the turbidity or chlorophyll content at constant level of the one or more algae strain of the an environmentally competitive algae population.
20. The method of statement 1-18 or 19, wherein measuring the photosynthetic efficiency and/or productivity of one or more algae strain of the an environmentally competitive algae population comprises measuring the ash free dry weight (AFDW) of the one or more algae strain of the an environmentally competitive algae population.
21. The method of statement 1-19 or 20, wherein isolating an environmentally competitive algae strain or a mixture of environmentally competitive algae strains that exhibit hybrid vigor under the selection conditions compared to at least one of the phenotypically-diverse algae strain(s) grown under baseline conditions comprises sequencing one or more segments of genomic DNA, cDNA, or RNA of an environmentally competitive algae strain or a mixture of environmentally competitive algae strains that exhibit hybrid vigor under the selection conditions.
22. The method of statement 21, further comprising isolating an environmentally competitive algae strain or a mixture of environmentally competitive algae strains that have one or more sequence differences in a segment of genomic DNA, cDNA, or RNA compared to the same segment of genomic DNA, cDNA, or RNA of at least one phenotypically-diverse algae strain grown under baseline conditions.
23. The method of statement 1-21 or 22, further comprising identifying one or more genomic locus that is (are) correlated with hybrid vigor under the selection conditions in an environmentally competitive algae strain or in a mixture of environmentally competitive algae strains.
24. The method of statement 1-22 or 23, further comprising pooling zygospores from one or more genetically diverse algae strains or from a mixture of genetically diverse algae strains, and hatching spores therefrom to generate a second genetically diverse strain population.
25. The method of statement 1-23 or 24, further comprising pooling zygospores from one or more environmentally competitive algae strain or from a mixture of environmentally competitive algae strains, and hatching spores therefrom to generate a second genetically diverse strain population.
26. The method of statement 1-24 or 25, wherein the phenotypically-diverse algae strains are sexually reproductive strains.
27. An environmentally competitive algae strain comprising at least one genomic locus, or at least two genomic loci, or at least three genomic loci, or at least four genomic loci, or at least five genomic loci that provide environmental competitiveness compared to a wild type algae or parental algae strain.
28. The environmentally competitive algae strain of statement 27, wherein the environmentally competitive algae strain has one or more genomic mutation compared to a wild type algae or parental algae strain at the least one genomic locus, the at least two genomic loci, the at least three genomic loci, the at least four genomic loci, or the at least five genomic loci that provide environmental competitiveness.
29. The environmentally competitive algae strain of statement 27 or 28, wherein the environmental competitiveness comprises enhanced growth of the environmentally competitive algae strain compared to the wild type algae or parental algae strain under conditions comprising an increased oxygen atmosphere, a reduced carbon dioxide atmosphere, reduced light conditions, increased light conditions, increased salt conditions, increased temperatures, decreased temperatures, fluctuating temperatures, reduced nitrogen conditions, reduced pH conditions, increased pH conditions, conditions comprising macronutrients, conditions comprising micronutrients, conditions comprising pollutants, reduced phosphate conditions, or increased phosphate conditions.
30. The environmentally competitive algae strain of statement 27, 28, or 29, wherein the environmental competitiveness comprises at least 2%, or at least 5%, or at least 10%, or at least 20%, or at least 25%, or at least 50%, or at least 75% enhanced growth of the environmentally competitive algae strain compared to the wild type algae or parental algae strain during culture for 1 to 30 days.
31. A population of algae comprising one or more of the environmentally competitive algae strain of statement 27, 28, 29, or 30.
32. The population of algae of statement 31, comprising at least 2%, or at least 5%, or at least 10%, or at least 20%, or at least 25%, or at least 50%, or at least 75%, or at least 80%, or at least 85%, or at least 90%, or at least 95%, or at least 97%, or at least 98%, or at least 99% algae of the environmentally competitive algae strain of statement 26, 27, 28, or 29.
33. A mixture of environmentally competitive algae strains, each environmentally competitive algae strain being the environmentally competitive algae strain of statement 27, 28, 29, or 30.
34. A genomic locus that confers environmental competitiveness to an algae strain, wherein the environmental competitiveness comprises enhanced growth of an algae strain with the genomic locus compared to a wild type algae or parental algae strain that does not comprised the genomic locus under conditions comprising an increased oxygen atmosphere, a reduced carbon dioxide atmosphere, reduced light conditions, increased light conditions, increased salt conditions, increased temperatures, decreased temperatures, fluctuating temperatures, reduced nitrogen conditions, reduced pH conditions, increased pH conditions, conditions comprising macronutrients, conditions comprising micronutrients, conditions comprising pollutants, reduced phosphate conditions, or increased phosphate conditions.
35. The genomic locus of statement 34, comprising one or more genomic mutation compared to the wild type algae or the parental algae strain at the genomic locus.

The specific methods, devices and compositions described herein are representative of preferred embodiments and are exemplary and not intended as limitations on the scope of the invention. Other objects, aspects, and embodiments will occur to those skilled in the art upon consideration of this specification, and are encompassed within the spirit of the invention as defined by the scope of the claims. It will be readily apparent to one skilled in the art that varying substitutions and modifications may be made to the invention disclosed herein without departing from the scope and spirit of the invention.

The invention illustratively described herein suitably may be practiced in the absence of any element or elements, or limitation or limitations, which is not specifically disclosed herein as essential. The methods and processes illustratively described herein suitably may be practiced in differing orders of steps, and the methods and processes are not necessarily restricted to the orders of steps indicated herein or in the claims.

Under no circumstances may the patent be interpreted to be limited to the specific examples or embodiments or methods specifically disclosed herein. Under no circumstances may the patent be interpreted to be limited by any statement made by any Examiner or any other official or employee of the Patent and Trademark Office unless such statement is specifically and without qualification or reservation expressly adopted in a responsive writing by Applicants.

The terms and expressions that have been employed are used as terms of description and not of limitation, and there is no intent in the use of such terms and expressions to exclude any equivalent of the features shown and described or portions thereof, but it is recognized that various modifications are possible within the scope of the invention as claimed. Thus, it will be understood that although the present invention has been specifically disclosed by preferred embodiments and optional features, modification and variation of the concepts herein disclosed may be resorted to by those skilled in the art, and that such modifications and variations are considered to be within the scope of this invention as defined by the appended claims and statements of the invention.

The invention has been described broadly and generically herein. Each of the narrower species and subgeneric groupings falling within the generic disclosure also form part of the invention. This includes the generic description of the invention with a proviso or negative limitation removing any subject matter from the genus, regardless of whether or not the excised material is specifically recited herein. In addition, where features or aspects of the invention are described in terms of Markush groups, those skilled in the art will recognize that the invention is also thereby described in terms of any individual member or subgroup of members of the Markush group.

What is claimed:

1. A method for producing algae with strong hybrid vigor for photosynthetic productivity comprising:
(a) crossing phenotypically-diverse algae strains to generate two or more genetically diverse algae strains;
(b) culturing one or more genetically diverse algae strain under one or more selection conditions to generate an environmentally competitive algae population;
(c) measuring the photosynthetic efficiency and/or productivity of one or more algae strain of the environmentally competitive algae population to produce one or more selected environmentally competitive algae strain; and
(d) isolating one or more environmentally competitive algae strain or a mixture of environmentally competitive algae strains that exhibit hybrid vigor under the selection conditions compared to at least one of the phenotypically-diverse algae strain(s) grown under baseline conditions.

2. The method of claim 1, wherein the selection conditions comprise an increased oxygen atmosphere, a reduced carbon dioxide atmosphere, reduced light conditions, increased light conditions, increased salt conditions, increased temperatures, decreased temperatures, fluctuating temperatures, reduced nitrogen conditions, reduced pH conditions, increased pH conditions, conditions comprising macronutrients, conditions comprising micronutrients, conditions comprising pollutants, reduced phosphate conditions, increased phosphate conditions, or a combination thereof.

3. The method of claim 1, wherein the baseline condition comprises 5% CO₂ in air, and a 14-hour light:10 dark cycle with zenith at noontime.

4. The method of claim 1, wherein the baseline condition comprises light intensity ascending to a zenith with maximum photosynthetically active radiation (PAR) of about 2000 μmol photons per square meter per second (m⁻²s⁻¹), and descending until dark, delivered in a sinusoidal form.

5. The method of claim 1, wherein one of the selection conditions comprises reduced carbon dioxide atmospheric conditions comprising an atmosphere of less than 0.04% CO₂.

6. The method of claim 1, wherein one of the selection conditions comprises reduced light stress conditions comprising cycles of 1-3 days of baseline light followed by 1-3 days of very low light.

7. The method of claim 1, wherein one of the selection conditions comprises reduced light stress conditions comprising:
a. one day of a baseline condition comprising 5% CO₂ in air, and a 14-hour light:10 dark cycle, wherein light intensity ascends at noon to a zenith with maximum photosynthetically active radiation (PAR) of about 2000 μmol photons per square meter per second (m⁻²s⁻¹), and descending until dark, delivered in a sinusoidal form; and
b. followed by three light starvation days, each light starvation day comprising a 14 hour: 10-hour light:dark, where the light comprises a rectangular wave with a PAR intensity of 50 μmol photons per square meter per second (m⁻²s⁻¹).

8. The method of claim 1, wherein one of the selection conditions comprises increased light conditions comprising more than 2000 μmol photons per square meter per second (m⁻²s⁻¹).

9. The method of claim 1, wherein one of the selection conditions comprises increased salt conditions comprising culturing the one or more genetically diverse algae strain in culture media comprising more than 0.2 M sodium chloride.

10. The method of claim 1, wherein one of the selection conditions comprises increased temperatures comprising culturing the one or more genetically diverse algae strain at more than 40° C.

11. The method of claim 1, wherein one of the selection conditions comprises decreased temperatures comprising culturing the one or more genetically diverse algae strain at less than 15° C.

12. The method of claim 1, wherein one of the selection conditions comprises fluctuating temperatures comprising culturing the one or more genetically diverse algae strain at fluctuating temperatures between 12° C. and 44° C.

13. The method of claim 1, wherein one of the selection conditions comprises reduced nitrogen conditions comprising culturing the one or more genetically diverse algae strain in culture media comprising less than 0.2 mM nitrate.

14. The method of claim 1, wherein one of the selection conditions comprises reduced phosphate conditions comprising culturing the one or more genetically diverse algae strain in culture media comprising less than 1 mM phosphate.

15. The method of claim 1, wherein one of the selection conditions comprises increased phosphate conditions comprising culturing the one or more genetically diverse algae strain in culture media comprising more than 2 mM phosphate.

16. The method of claim 1, wherein at least one of the phenotypically-diverse algae strain(s) is a species of Protococcus, Ulva, Codium, Enteromorpha, Neochloris and/or Chlamydomonas.

17. The method of claim 1, wherein at least one of the phenotypically-diverse algae strain(s) is a Chlamydomonas reinhardtii strain.

18. The method of claim 1, wherein measuring the photosynthetic efficiency and/or productivity of one or more algae strain of the an environmentally competitive algae population comprises measuring the number of daily dilutions needed to maintain the turbidity or chlorophyll content of the one or more algae strain culture at a constant level.

19. The method of claim 1, wherein measuring the photosynthetic efficiency and/or productivity of one or more algae strain of the an environmentally competitive algae population comprises measuring the ash free dry weight (AFDW) of the one or more algae strain of the an environmentally competitive algae population.

20. The method of claim 1, further comprising isolating an environmentally competitive algae strain or a mixture comprises sequencing one or more segments of genomic DNA, cDNA, or RNA of an environmentally competitive algae strain or a mixture of environmentally competitive algae strains that exhibit hybrid vigor under the selection conditions.

21. The method of claim 20, further comprising isolating an environmentally competitive algae strain or a mixture of environmentally competitive algae strains that have one or more sequence differences in a segment of genomic DNA, cDNA, or RNA compared to the same segment of genomic DNA, cDNA, or RNA of at least one phenotypically-diverse algae strain grown under baseline conditions.

22. The method of claim 1, further comprising identifying one or more genomic locus that is correlated with hybrid vigor under the selection conditions in an environmentally competitive algae strain or in a mixture of environmentally competitive algae strains.

23. The method of claim 1, further comprising pooling zygospores from one or more genetically diverse algae strains or from a mixture of genetically diverse algae strains, and hatching spores therefrom to generate a second genetically diverse strain population.

24. The method of claim 23, further comprising pooling zygospores from one or more strain of the second genetically diverse strain population, and hatching spores therefrom to generate a third genetically diverse strain population.

25. The method of claim 1, wherein the phenotypically-diverse algae strains are sexually reproductive strains.

26. An environmentally competitive algae strain produced by the method of claim 1.

27. An environmentally competitive algae strain of claim 26, comprising at least one genomic locus, or at least two genomic loci, or at least three genomic loci, or at least four genomic loci, or at least five genomic loci that provide environmental competitiveness over a wild-type algae or over a parental algae strain of the environmentally competitive algae strain.

28. The environmentally competitive algae strain of claim 27, wherein the environmentally competitive algae strain has one or more genomic mutation compared to a wild type algae or parental algae strain within the at the least one genomic locus, the at least two genomic loci, the at least three genomic loci, the at least four genomic loci, or the at least five genomic loci that provide environmental competitiveness.

29. The environmentally competitive algae strain of claim 27, wherein the environmental competitiveness comprises enhanced growth of the environmentally competitive algae strain compared to the wild type algae or parental algae strain under conditions comprising an increased oxygen atmosphere, a reduced carbon dioxide atmosphere, reduced light conditions, increased light conditions, increased salt conditions, increased temperatures, decreased temperatures, fluctuating temperatures, reduced nitrogen conditions, reduced pH conditions, increased pH conditions, conditions comprising macronutrients, conditions comprising micronutrients, conditions comprising pollutants, reduced phosphate conditions, or increased phosphate conditions.

30. The environmentally competitive algae strain of claim 27, wherein the environmental competitiveness comprises at least 5% enhanced growth of the environmentally competitive algae strain compared to the wild type algae or parental algae strain during culture for 1 to 30 days.

31. A population of algae comprising one or more of the environmentally competitive algae strains of claim 26.

32. A genomic locus that confers environmental competitiveness to an algae strain, wherein the environmental competitiveness comprises enhanced growth of an algae strain with the genomic locus compared to a wild type algae or parental algae strain that does not comprised the genomic locus under conditions comprising an increased oxygen atmosphere, a reduced carbon dioxide atmosphere, reduced light conditions, increased light conditions, increased salt conditions, increased temperatures, decreased temperatures, fluctuating temperatures, reduced nitrogen conditions, reduced pH conditions, increased pH conditions, conditions comprising macronutrients, conditions comprising micronutrients, conditions comprising pollutants, reduced phosphate conditions, or increased phosphate conditions.

33. The genomic locus of claim 32, comprising one or more genomic mutation compared to the wild type algae or the parental algae strain at the genomic locus.

34. A method for producing algae with strong hybrid vigor for photosynthetic productivity comprising:
a. mating two phenotypically-diverse algae strains to generate two or more genetically diverse algae strains;
b. culturing one or more genetically diverse algae strain under one or more selection conditions to generate an environmentally competitive algae population, where the selection conditions comprise:
i. hyperoxic atmospheric conditions comprising 5% CO₂ in oxygen;
ii. light stress conditions comprising alternating one day of 2000 μmol photons light per square meter per second (m⁻²s⁻¹) and then three days of 50 μmol photons light per square meter per second (m⁻²s⁻¹); or
iii. high salt conditions comprising culturing in a medium comprising 20 g/L of Instant Ocean salts,
c. measuring the photosynthetic efficiency of one or more algae strain of the an environmentally competitive algae population; and
d. isolating an environmentally competitive algae strain or a mixture of environmentally competitive algae strains that exhibit hybrid vigor under the selection conditions compared to at least one of the phenotypically-diverse algae strain(s) grown under baseline conditions.
